(19) United States
(12) Patent Application Publication
(10) Pub. No.: US 2019/0378611 A1
(43) Pub. Date: Dec. 12, 2019

(54) HEALTHCARE PROGRAM MANAGEMENT METHOD AND ELECTRONIC DEVICE THEREOF

(71) Applicant: Samsung Electronics Co., Ltd., Suwon-si, Gyeonggi-do (KR)

(72) Inventors: Jeong-Ja KIM, Suwon-si (KR); Byungwoan KIM, Yongin-si (KR); Jaeyoung KIM, Seoul (KR); Kyung Sub MIN, Seongnam-si (KR); Heeseung SHIN, Seoul (KR); Woong Jo WOO, Seoul (KR)

(21) Appl. No.: 16/481,414

(22) PCT Filed: Jan. 15, 2018
(86) PCT No.: PCT/KR2018/000683

§ 371 Date: Jul. 26, 2019

(30) Foreign Application Priority Data
Jan. 26, 2017 (KR) 10-2017-0012669

(51) Int. Cl. G16H 40/63; G16H 20/30

(52) U.S. Cl. CPC G16H 40/63; G16H 20/30

(57) ABSTRACT

A method according to various embodiments of the present invention can comprise operations of: receiving a first healthcare program from another electronic device; generating a first schedule including at least one activity on the basis of the received first healthcare program; providing notification to a user on the basis of the first schedule; sensing an activity of the user related to a first activity during the at least one activity; linking, with the first activity, a first item included in an exercise application associated with the first activity; and reflecting, in the first item, user activity information related to the first activity.

### CROSS-REFERENCE TO RELATED APPLICATIONS

This application is a 371 National Stage of International Application No. PCT/KR2018/000683, filed Jan. 15, 2018, which claims priority to Korean Patent Application No. 10-2017-0012669, filed Jan. 26, 2017, the disclosures of which are herein incorporated by reference in their entirety.

### BACKGROUND

### 1. Field

Various embodiments of the disclosure relate to a healthcare program management method and an electronic device supporting the same.

### 2. Description of Related Art

With the development of information communication technology and semiconductor technology, various electronic devices are being developed as multimedia devices capable of providing various multimedia services. For example, portable electronic devices can provide a variety of multimedia services such as broadcast services, wireless Internet services, camera services, and music playback services.

The electronic device may provide a healthcare program to meet the interest of users in caring for their health. For example, the electronic device may provide a user with a healthcare program that includes a sequence and method of movement, such as rope skipping, muscular motion, etc., through an exercise application.

When the electronic device provides the healthcare program, the user of the electronic device must manually input an activity result in performing an activity included in the healthcare program, it is difficult to receive expert feedback according to the activity result, and there is an inconvenience in that a recommended healthcare program based on user information and experience cannot be provided.

Various embodiments of the disclosure may provide a healthcare program management method and an electronic device supporting the same.

The technical subjects pursued in the disclosure may not be limited to the above mentioned technical subjects, and other technical subjects which are not mentioned may be clearly understood, through the following descriptions, by those skilled in the art of the disclosure.

### SUMMARY

In accordance with an aspect of the present invention, there is provided a method according to various embodiments of the disclosure, including: receiving a first healthcare program from another electronic device; generating a first schedule including at least one activity based on the received first healthcare program; providing a notification to a user based on the first schedule; sensing the activity of the user related to a first activity during the at least one activity; linking, with the first activity, a first item included in an exercise application associated with the first activity; and reflecting, in the first item, activity information of the user related to the first activity.

In accordance with another aspect of the present invention, there is provided a method according to various embodiments of the disclosure, including: receiving an input of healthcare program information; determining whether an input of session information is detected; determining whether an activity of a user is detected when the input of the session information is not detected; generating the session information based on the activity of the user when an activity of a provider is detected; and generating a healthcare program based on the healthcare program information and the session information.

An electronic device according to various embodiments of the disclosure may include: a communication module; a sensor module; a processor configured to be electrically connected to the communication module and the sensor module; and a memory configured to be electrically connected to the processor, wherein the memory is controlled to store, when executed, instructions that cause the processor to receive a first healthcare program from at least one another electronic device using the communication module, to generate a first schedule including at least one activity based on the received first healthcare program, to provide a notification to a user based on the first schedule, to sense an activity of the user related to a first activity during the at least one activity, using the sensor module, to link, with the first activity, a first item included in an exercise application associated with the first activity, to reflect, in the first item, activity information of the user related to the first activity acquired using the sensor module.

An electronic device according to various embodiments of the disclosure may include: a sensor configured to be functionally connected to the electronic device; and a processor, wherein the processor receives an input of healthcare program information, determines whether an input of session information is detected, determines whether an activity of a user is detected when the input of the session information is not detected, generates the session information based on the activity of the user when the activity of the user is detected, and generates a healthcare program based on the healthcare program information and the session information.

A method according to various embodiments of the disclosure and an electronic device supporting the same can easily generate and distribute various healthcare programs pertaining to, for example, running, weight management, or skincare.

In addition, a method according to various embodiments of the disclosure and an electronic device supporting the same can record activity content of a user by linking a healthcare program distributed in the electronic device with an exercise application.

In addition, a method according to various embodiments of the disclosure and an electronic device supporting the same can provide user's healthcare program progress information to a server, and can receive the updated healthcare program and expert's services from the server.

In addition, a method according to various embodiments of the disclosure and an electronic device supporting the same can provide a recommended healthcare program list based on user information and user experience.

In addition, a method according to various embodiments of the disclosure and an electronic device supporting the same can interwork with a wearable device to record the activity information of a user.

### BRIEF DESCRIPTION OF THE DRAWINGS

FIG. 1 illustrates an environment for a network including an electronic device according to various embodiments of the disclosure;

FIG. 2 is a block diagram illustrating an electronic device according to various embodiments of the disclosure;

FIG. 3 is a block diagram illustrating a healthcare program module according to various embodiments;

FIG. 4 is a block diagram illustrating the configuration of an electronic device and a server according to various embodiments of the disclosure;

FIG. 5 is a flowchart illustrating an operation method of an electronic device according to various embodiments of the disclosure;

FIGS. 6A to 6C are exemplary diagrams illustrating an exercise application interworking with a healthcare program according to an embodiment of the disclosure;

FIGS. 7A and 7B are exemplary diagrams illustrating a method of displaying an alarm according to an embodiment of the disclosure;

FIG. 8 is an exemplary diagram illustrating a method of displaying a healthcare program according to an embodiment of the disclosure;

FIG. 9 is an exemplary diagram illustrating a method of performing a healthcare program according to an embodiment of the disclosure;

FIG. 10 is an exemplary diagram illustrating a method of confirming the progress of a healthcare program according to an embodiment of the disclosure;

FIG. 11 is a flowchart illustrating a system between an electronic device according to an embodiment of the disclosure and a server;

FIG. 12 is a flowchart illustrating an operation method of an electronic device according to an embodiment of the disclosure;

FIG. 13 is a flowchart illustrating a method of generating a healthcare program according to an embodiment of the disclosure;

FIGS. 14A and 14B are exemplary diagrams illustrating a method of generating a healthcare program according to an embodiment of the disclosure;

FIG. 15 is an exemplary diagram illustrating a method of generating a healthcare program according to an embodiment of the disclosure;

FIG. 16 is a flowchart illustrating an operation method of a server according to an embodiment of the disclosure;

FIG. 17 is a flowchart illustrating a system among a first electronic device, a server, and a second electronic device according to an embodiment of the disclosure; and

FIG. 18 is a flowchart illustrating a system between an electronic device according to an embodiment of the disclosure and a wearable device.

### DETAILED DESCRIPTION

Hereinafter, various exemplary embodiments of the present document are described with reference to the accompanying drawings. It should be understood, however, that it is not intended to limit the various exemplary embodiments of the present document to the particular form disclosed, but, on the contrary, it is intended to cover all modifications, equivalents, and alternatives falling within the spirit and scope of the various exemplary embodiments of the present document. Like reference numerals denote like components throughout the drawings. A singular expression includes a plural concept unless there is a contextually distinctive difference therebetween. In the present document, an expression “A or B”, “A and/or B”, or the like may include all possible combinations of items enumerated together. Although expressions such as “1st”, “2nd”, “first”, and “second” may be used to express corresponding constitutional elements, it is not intended to limit the corresponding constitutional elements. When a certain (e.g., 1st) constitutional element is mentioned as being “operatively or communicatively coupled with/to” or “connected to” a different (e.g., 2nd) constitutional element, the certain constitutional element is directly coupled with/to another constitutional element or can be coupled with/to the different constitutional element via another (e.g., 3rd) constitutional element.

An expression “configured to” used in the present document may be interchangeably used with, for example, “suitable for”, “having the capacity to”, “adapted to”, “made to”, “capable of”, or “designed to” in a hardware or software manner according to a situation. In a certain situation, an expressed “a device configured to” may imply that the device is “capable of” together with other devices or components. For example, “a processor configured to perform A, B, and C” may imply a dedicated processor (e.g., an embedded processor) for performing a corresponding operation or a generic-purpose processor (e.g., Central Processing Unit (CPU) or an application processor) capable of performing corresponding operations by executing one or more software programs stored in a memory device.

An electronic device according to various embodiments of the present disclosure, for example, may include at least one of a smartphone, a tablet personal computer (PC), a mobile phone, a video phone, an electronic book (e-book) reader, a desktop PC, a laptop PC, a netbook computer, a workstation, a server, a personal digital assistant (PDA), a portable multimedia player (PMP), an MP3 player, a mobile medical appliance, a camera, and a wearable device (e.g., smart glasses, a head-mounted-device (HIVID), electronic clothes, an electronic bracelet, an electronic necklace, an electronic appcessory, an electronic tattoo, a smart mirror, or a smart watch). According to some embodiments, the electronic device (ex. home appliance) may include at least one of, for example, a television, a Digital Video Disk (DVD) player, an audio, a refrigerator, an air conditioner, a vacuum cleaner, an oven, a microwave oven, a washing machine, an air cleaner, a set-top box, a home automation control panel, a security control panel, a TV box (e.g., Samsung HomeSync™, Apple TV′, or Google TV^{Tm}), a game console (e.g., Xbox™ and PlayStation™), an electronic dictionary, an electronic key, a camcorder, and an electronic photo frame.

According to another embodiment, the electronic device may include at least one of various medical devices (e.g., various portable medical measuring devices (a blood glucose monitoring device, a heart rate monitoring device, a blood pressure measuring device, a body temperature measuring device, etc.), a Magnetic Resonance Angiography (MRA), a Magnetic Resonance Imaging (MM), a Computed Tomography (CT) machine, and an ultrasonic machine), a navigation device, a Global Positioning System (GPS) receiver, an Event Data Recorder (EDR), a Flight Data Recorder (FDR), a Vehicle Infotainment Devices, an electronic devices for a ship (e.g., a navigation device for a ship, and a gyro-compass), avionics, security devices, an automotive head unit, a robot for home or industry, an automatic teller's machine (ATM) in banks, point of sales (POS) in a shop, or internet device of things (e.g., a light bulb, various sensors, electric or gas meter, a sprinkler device, a fire alarm, a thermostat, a streetlamp, a toaster, a sporting goods, a hot water tank, a heater, a boiler, etc.). According to some embodiments, the electronic device may include at least one of a part of furniture or a building/structure, an electronic board, an electronic signature receiving device, a projector, and various kinds of measuring instruments (e.g., a water meter, an electric meter, a gas meter, and a radio wave meter). The electronic device according to various embodiments of the present disclosure may be a combination of one or more of the aforementioned various devices. The electronic device according to some embodiments of the present disclosure may be a flexible device. Further, the electronic device according to an embodiment of the present disclosure is not limited to the aforementioned devices, and may include a new electronic device according to the development of technology. Hereinafter, an electronic device according to various embodiments will be described with reference to the accompanying drawings. As used herein, the term “user” may indicate a person who uses an electronic device or a device (e.g., an artificial intelligence electronic device) that uses an electronic device.

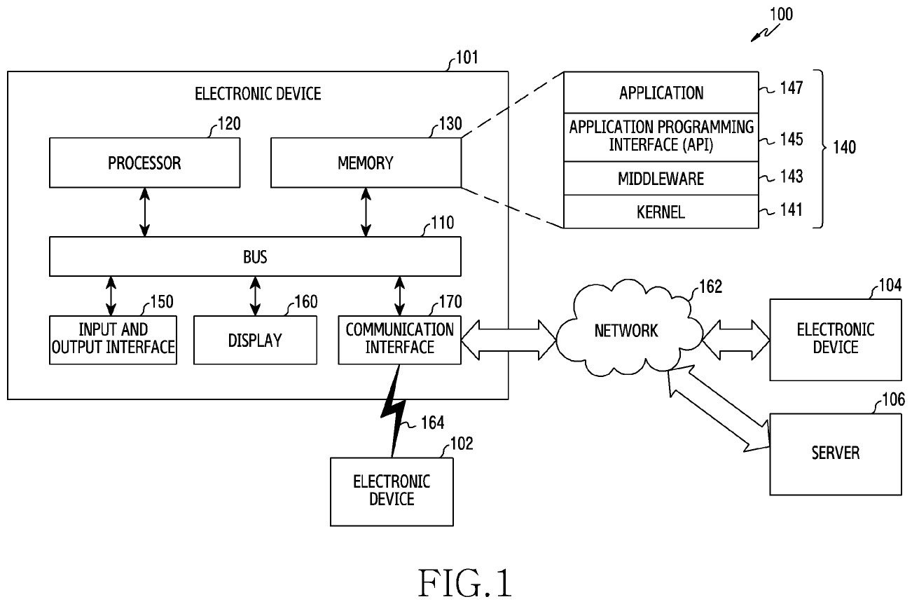

FIG. 1 is a diagram illustrating an example network environment including an electronic device according to various example embodiments of the present disclosure.

An electronic device 101 within a network environment 100, according to various embodiments, will be described with reference to FIG. 1. The electronic device 101 may include a bus 110, a processor (e.g., including processing circuitry) 120, a memory 130, an input/output interface (e.g., including input/output circuitry) 150, a display 160, a communication interface (e.g., including communication circuitry) 170 and a handle 180. According to an example embodiment of the present disclosure, the electronic device 101 may omit at least one of the above components or may further include other components. The bus 110 may include, for example, a circuit which interconnects the components 110 to 170 and delivers a communication (e.g., a control message and/or data) between the components 110 to 170. The processor 120 may include various processing circuitry, such as, for example, and without limitation, one or more of a dedicated processor, a Central Processing Unit (CPU), an Application Processor (AP), and a Communication Processor (CP). The processor 120 may carry out, for example, calculation or data processing relating to control and/or communication of at least one other component of the electronic device 101.

The memory 130 may include a volatile memory and/or a non-volatile memory. The memory 130 may store, for example, commands or data relevant to at least one other component of the electronic device 101. According to an embodiment of the present disclosure, the memory 130 may store software and/or a program 140.

The program 140 may include, for example, a kernel 141, middleware 143, an Application Programming Interface (API) 145, and/or application programs (or “applications”) 147. At least some of the kernel 141, the middleware 143, and the API 145 may be referred to as an Operating System (OS). The memory 130 may include a computer readable recording medium having a program recorded thereon to execute the method according to various example embodiments in the processor 120. The kernel 141 may control or manage system resources (e.g., the bus 110, the processor 120, or the memory 130) used for performing an operation or function implemented in the other programs (e.g., the middleware 143, the API 145, or the application programs 147). Furthermore, the kernel 141 may provide an interface through which the middleware 143, the API 145, or the application programs 147 may access the individual components of the electronic device 101 to control or manage the system resources.

The middleware 143, for example, may serve as an intermediary for allowing the API 145 or the application programs 147 to communicate with the kernel 141 to exchange data. Also, the middleware 143 may process one or more task requests received from the application programs 147 according to priorities thereof. For example, the middleware 143 may assign priorities for using the system resources (e.g., the bus 110, the processor 120, the memory 130, or the like) of the electronic device 101, to at least one of the application programs 147. For example, the middleware 143 may perform scheduling or loading balancing on the one or more task requests by processing the one or more task requests according to the priorities assigned thereto. The API 145 is an interface through which the applications 147 control functions provided from the kernel 141 or the middleware 143, and may include, for example, at least one interface or function (e.g., instruction) for file control, window control, image processing, character control, and the like. The input/output interface 150, for example, may include various input/output circuitry and function as an interface that may transfer commands or data input from a user or another external device to the other element(s) of the electronic device 101. Furthermore, the input/output interface 150 may output the commands or data received from the other element(s) of the electronic device 101 to the user or another external device.

Examples of the display 160 may include a Liquid Crystal Display (LCD), a Light-Emitting Diode (LED) display, an Organic Light-Emitting Diode (OLED) display, a MicroElectroMechanical Systems (MEMS) display, and an electronic paper display, or the like, but is not limited thereto. The display 160 may display, for example, various types of contents (e.g., text, images, videos, icons, or symbols) to users. The display 160 may include a touch screen, and may receive, for example, a touch, gesture, proximity, or hovering input using an electronic pen or a user's body part

The communication interface 170 may include various communication circuitry and may establish communication, for example, between the electronic device 101 and an external device (e.g., a first external electronic device 102, a second external electronic device 104, or a server 106). For example, the communication interface 170 may be connected to a network 162 through wireless or wired communication, and may communicate with an external device (e.g., the second external electronic device 104 or the server 106).

The wireless communication may use at least one of, for example, Long Term Evolution (LTE), LTE-Advance (LTE-A), Code Division Multiple Access (CDMA), Wideband CDMA (WCDMA), Universal Mobile Telecommunications System (UMTS), Wireless Broadband (WiBro), and Global System for Mobile Communications (GSM), as a cellular communication protocol. In addition, the wireless communication may include, for example, short range communication 164. The short-range communication 164 may include at least one of, for example, Wi-Fi, Bluetooth, Near Field Communication (NFC), and Global Navigation Satellite System (GNSS). GNSS may include, for example, at least one of global positioning system (GPS), global navigation satellite system (Glonass), Beidou Navigation satellite system (Beidou) or Galileo, and the European global satellite-based navigation system, based on a location, a bandwidth, or the like. Hereinafter, in the present disclosure, the “GPS” may be interchangeably used with the “GNSS”. The wired communication may include, for example, at least one of a Universal Serial Bus (USB), a High Definition Multimedia Interface (HDMI), Recommended Standard 232 (RS-232), and a Plain Old Telephone Service (POTS).

The network 162 may include at least one of a telecommunication network such as a computer network (e.g., a LAN or a WAN), the Internet, and a telephone network.

Each of the first and second external electronic devices 102 and 104 may be of a type identical to or different from that of the electronic device 101. According to an embodiment of the present disclosure, the server 106 may include a group of one or more servers. According to various example embodiments of the present disclosure, all or some of the operations performed in the electronic device 101 may be executed in another electronic device or a plurality of electronic devices (e.g., the electronic devices 102 and 104 or the server 106). According to an embodiment of the present disclosure, when the electronic device 101 has to perform some functions or services automatically or in response to a request, the electronic device 101 may request another device (e.g., the electronic device 102 or 104 or the server 106) to execute at least some functions relating thereto instead of or in addition to autonomously performing the functions or services. Another electronic device (e.g., the electronic device 102 or 104, or the server 106) may execute the requested functions or the additional functions, and may deliver a result of the execution to the electronic device 101. The electronic device 101 may process the received result as it is or additionally, and may provide the requested functions or services. To this end, for example, cloud computing, distributed computing, or client-server computing technologies may be used.

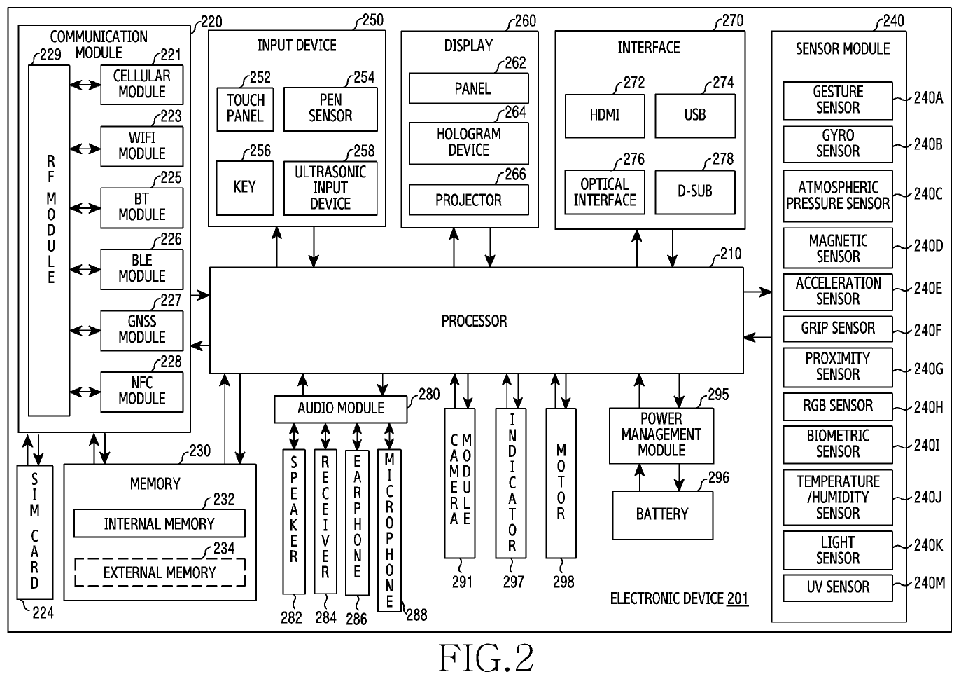

FIG. 2 is a block diagram illustrating an example electronic device according to various example embodiments of the present disclosure.

The electronic device 201 may include, for example, all or a part of the electronic device 101 illustrated in FIG. 1. The electronic device 201 may include one or more processors (e.g., including processing circuitry) 210 (e.g., Application Processors (AP)), a communication module (e.g., including communication circuitry) 220, a Subscriber Identification Module (SIM) 224, a memory 230, a sensor module 240, an input device (e.g., including input circuitry) 250, a display 260, an interface (e.g., including interface circuitry) 270, an audio module 280, a camera module (e.g., including a camera) 291, a power management module 295, a battery 296, an indicator 297, and a motor 298.

The processor 210 may include various processing circuitry configured to control a plurality of hardware or software components connected to the processor 210 by driving an operating system or an application program, and perform processing of various pieces of data and calculations. The processor 210 may be embodied as, for example, a System on Chip (SoC). According to an embodiment of the present disclosure, the processor 210 may further include a Graphic Processing Unit (GPU) and/or an image signal processor. The processor 210 may include at least some (for example, a cellular module 221) of the components illustrated in FIG. 2. The processor 210 may load, into a volatile memory, commands or data received from at least one (e.g., a non-volatile memory) of the other components and may process the loaded commands or data, and may store various data in a non-volatile memory.

The communication module 220 may have a configuration equal or similar to that of the communication interface 170 of FIG. 1. The communication module 220 may include various communication circuitry, such as, for example, and without limitation, a cellular module 221, a Wi-Fi module 223, a BT module 225, a GNSS module 227 (e.g., a GPS module 227, a Glonass module, a Beidou module, or a Galileo module), an NFC module 228, and a Radio Frequency (RF) module 229. The cellular module 221, for example, may provide a voice call, a video call, a text message service, or an Internet service through a communication network. According to an embodiment of the present disclosure, the cellular module 221 may distinguish and authenticate the electronic device 201 in a communication network using the subscriber identification module 224 (for example, the SIM card). According to an embodiment of the present disclosure, the cellular module 221 may perform at least some of the functions that the AP 210 may provide. According to an embodiment of the present disclosure, the cellular module 221 may include a communication processor (CP). For example, each of the Wi-Fi module 223, the BT module 225, the GNSS module 227, and the NFC module 228 may include a processor for processing data transmitted/received through a corresponding module. According to an embodiment of the present disclosure, at least some (e.g., two or more) of the cellular module 221, the Wi-Fi module 223, the BT module 225, the GNSS module 227, and the NFC module 228 may be included in one Integrated Chip (IC) or IC package. The RF module 229, for example, may transmit/receive a communication signal (e.g., an RF signal). The RF module 229 may include, for example, a transceiver, a Power Amplifier Module (PAM), a frequency filter, a Low Noise Amplifier (LNA), and an antenna. According to another embodiment of the present disclosure, at least one of the cellular module 221, the WIFI module 223, the BT module 225, the GNSS module 227, and the NFC module 228 may transmit/receive an RF signal through a separate RF module. The subscriber identification module 224 may include, for example, a card including a subscriber identity module and/or an embedded SIM, and may contain unique identification information (e.g., an Integrated Circuit Card Identifier (ICCID)) or subscriber information (e.g., an International Mobile Subscriber Identity (IMSI)).

The memory 230 (e.g., the memory 130) may include, for example, an embedded memory 232 and/or an external memory 234. The embedded memory 232 may include at least one of a volatile memory (e.g., a Dynamic Random Access Memory (DRAM), a Static RAM (SRAM), a Synchronous Dynamic RAM (SDRAM), and the like) and a non-volatile memory (e.g., a One Time Programmable Read Only Memory (OTPROM), a Programmable ROM (PROM), an Erasable and Programmable ROM (EPROM), an Electrically Erasable and Programmable ROM (EEPROM), a mask ROM, a flash ROM, a flash memory (e.g., a NAND flash memory or a NOR flash memory), a hard disc drive, a Solid State Drive (SSD), and the like). The external memory 234 may further include a flash drive, for example, a Compact Flash (CF), a Secure Digital (SD), a Micro Secure Digital (Micro-SD), a Mini Secure Digital (Mini-SD), an eXtreme Digital (xD), a MultiMediaCard (MMC), a memory stick, or the like. The external memory 234 may be functionally and/or physically connected to the electronic device 201 through various interfaces.

The sensor module 240, for example, may measure a physical quantity or detect an operation state of the electronic device 201, and may convert the measured or detected information into an electrical signal. The sensor module 240 may include, for example, at least one of a gesture sensor 240A, a gyro sensor 240B, an atmospheric pressure sensor (barometer) 240C, a magnetic sensor 240D, an acceleration sensor 240E, a grip sensor 240F, a proximity sensor 240G, a color sensor 240H (e.g., red, green, and blue (RGB) sensor), a biometric sensor (medical sensor) 240I, a temperature/humidity sensor 240I, an illuminance (e.g., light) sensor 240K, and a Ultra Violet (UV) sensor 240M. Additionally or alternatively, the sensor module 240 may include, for example, an E-nose sensor, an electromyography (EMG) sensor, an electroencephalogram (EEG) sensor, an electrocardiogram (ECG) sensor, an Infrared (IR) sensor, an iris scan sensor, and/or a finger scan sensor. The sensor module 240 may further include a control circuit for controlling one or more sensors included therein. According to an embodiment of the present disclosure, the electronic device 201 may further include a processor configured to control the sensor module 240, as a part of the processor 210 or separately from the processor 210, and may control the sensor module 240 while the processor 210 is in a sleep state. It is possible to detect a user wearing a head-mounted electronic device using the gyro sensor 240B, the acceleration sensor 240E, the geomagnetic sensor 240P, the proximity sensor 240G, or the grip sensor 240F.

According to an embodiment of the disclosure, the head-mounted device 201 may detect at least one of IR recognition, pressure recognition, and variation in capacitance (or dielectric constant) due to being worn by a user, thereby detecting whether the user is wearing the head-mounted device 201. The gesture sensor 240A may detect the movement of the user's hand or finger to receive the detected movement as an input operation of the head-mounted device 201. Additionally or alternatively, the sensor module 240 may use a biometric recognition sensor, for example, an E-nose sensor, an electromyography (EMG) sensor, an electroencephalogram (EEG) sensor, an electrocardiogram (ECG) sensor, an iris sensor, a refraction sensor, or the like to recognize biometric information of the user. The sensor module 240A may further include a control circuit for controlling at least one or more sensors belonging to the sensor module 240A.

The input device 250 may include various input circuitry, such as, for example, and without limitation, a touch panel 252, a (digital) pen sensor 254, a key 256, or an ultrasonic input device 258. The touch panel 252 may use, for example, at least one of a capacitive type, a resistive type, an infrared type, and an ultrasonic type. The touch panel 252 may further include a control circuit. The touch panel 252 may further include a tactile layer, and provide a tactile reaction to the user. The (digital) pen sensor 254 may include, for example, a recognition sheet which is a part of the touch panel or is separated from the touch panel. The key 256 may include, for example, a physical button, an optical key or a keypad. The ultrasonic input device 258 may detect, through a microphone (e.g., the microphone 288), ultrasonic waves generated by an input tool, and identify data corresponding to the detected ultrasonic waves.

The display 260 (e.g., the display 160) may include a panel 262, a hologram device 264, or a projector 266. The panel 262 may be implemented to be, for example, flexible, transparent, or wearable. The panel 262 may be embodied as a single module with the touch panel 252. The panel 262 may include a liquid crystal display (LCD), an organic light-emitting diode (OLED), an electronic ink, or an electro wetting display (EWD). The display 260 may have the property of transmitting light (e.g., a display having a light transmittance). For example, the display 260 having the light transmittance may be implemented in a manner such that a plurality of transparent or semi-transparent regions capable of emitting light is arranged together with pixels. Alternatively, the display 260 having the light transmittance may be implemented in a manner such that a plurality of through holes capable of transmitting light is arranged together with pixels. The hologram device 264 may show a three dimensional (3D) image in the air by using an interference of light. The projector 266 may project light onto a screen to display an image. The screen may be located, for example, in the interior of or on the exterior of the electronic device 201. The interface 270 may include various interface circuitry, such as, for example, and without limitation, a High-Definition Multimedia Interface (HDMI) 272, a Universal Serial Bus (USB) 274, an optical interface 276, or a D-subminiature (D-sub) 278. The interface 270 may be included in, for example, the communication interface 170 illustrated in FIG. 1. Additionally or alternatively, the interface 270 may include, for example, a Mobile High-definition Link (MHL) interface, a Secure Digital (SD) card/Multi-Media Card (MMC) interface, or an Infrared Data Association (IrDA) standard interface.

The audio module 280, for example, may bilaterally convert a sound and an electrical signal. At least some components of the audio module 280 may be included in, for example, the input/output interface 150 illustrated in FIG. 1. The audio module 280 may process voice information input or output through, for example, a speaker 282, a receiver 284, earphones 286, or the microphone 288.

The camera module 291 may include various circuitry including, for example, and without limitation, a camera, a device which may photograph a still image and a video, or the like. According to an embodiment of the present disclosure, the camera module 291 may include one or more image sensors (e.g., a front sensor or a back sensor), a lens, an Image Signal Processor (ISP) or a flash (e.g., LED or xenon lamp). The power management module 295 may manage, for example, power of the electronic device 201. According to an embodiment of the present disclosure, the power management module 295 may include a Power Management Integrated Circuit (PMIC), a charger Integrated Circuit (IC), or a battery or fuel gauge. The PMIC may use a wired and/or wireless charging method. Examples of the wireless charging method may include, for example, a magnetic resonance method, a magnetic induction method, an electromagnetic wave method, and the like. Additional circuits (e.g., a coil loop, a resonance circuit, a rectifier, etc.) for wireless charging may be further included. The battery gauge may measure, for example, a residual quantity of the battery 296, and a voltage, a current, or a temperature while charging. The battery 296 may include, for example, a rechargeable battery and/or a solar battery.

The indicator 297 may display a particular state (e.g., a booting state, a message state, a charging state, or the like) of the electronic device 201 or a part (e.g., the processor 210) of the electronic device 201. The motor 298 may convert an electrical signal into a mechanical vibration, and may generate a vibration, a haptic effect, or the like. Although not illustrated, the electronic device 201 may include a processing device (e.g., a GPU) for supporting a mobile TV. The processing device for supporting a mobile TV may process, for example, media data according to a certain standard such as Digital Multimedia Broadcasting (DMB), Digital Video Broadcasting (DVB), or mediaFLO™.

The gaze tracking unit 294 may track the user's gaze using, for example, at least one of an electrical oculography (EOG) sensor, a coil system, a dual purkinje system, a bright pupil system, and a dark pupil system. The gaze tracking unit 294 may further include a micro camera for gaze tracking.

The lens position adjustment unit 299 may arrange the lens so as to overlap the display 260 so that a user can see the image of the display 260 through the lens. Alternatively, the lens position adjustment unit 260 may move the lens to a position where the lens does not overlap the display 260 when the display 260 is in an inactive state. Alternatively, the lens position adjustment unit 299 may adjust the position of the lens so that the user can enjoy an image suitable for his or her own visual acuity. For example, the lens position adjustment unit 299 may perform a refraction test on the user's eyes and may adjust the position of the lens according to the result of the examination. Alternatively, the lens position adjustment unit 299 may adjust the position of the lens by measuring an inter-pupil distance (IPD) of the user.

Each of the above-described component elements of hardware according to the present disclosure may be configured with one or more components, and the names of the corresponding component elements may vary based on the type of electronic device. In various embodiments, the electronic device may include at least one of the above-described elements. Some of the above-described elements may be omitted from the electronic device, or the electronic device may further include additional elements. Also, some of the hardware components according to various embodiments may be combined into one entity, which may perform functions identical to those of the relevant components before the combination.

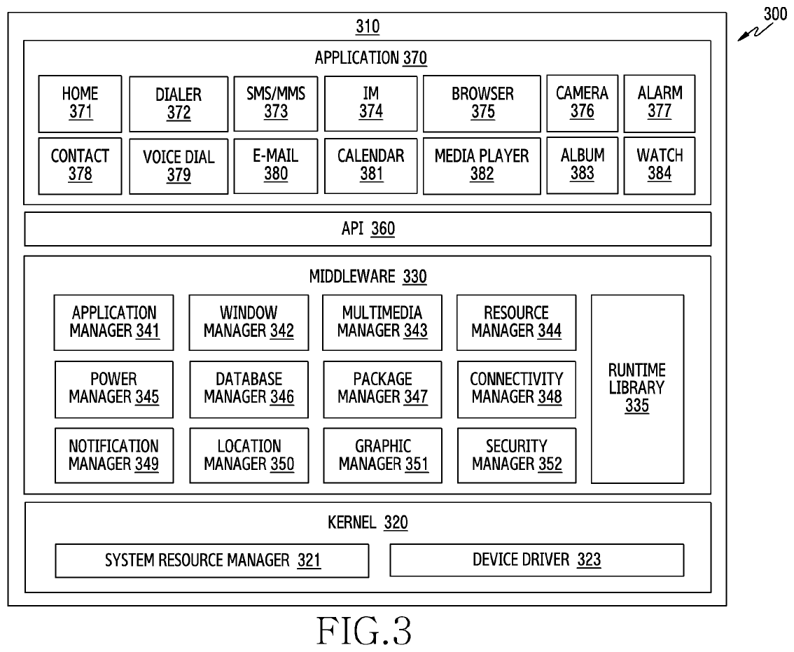

FIG. 3 is a block diagram illustrating an example program module according to various example embodiments of the present disclosure.

According to an embodiment of the present disclosure, the program module 310 (e.g., the program 140) may include an Operating System (OS) for controlling resources related to the electronic device (e.g., the electronic device 101) and/or various applications (e.g., the application programs 147) executed in the operating system. The operating system may be, for example, Android™, iOS™, Windows™, Symbian™, Tizen™, Bada™, or the like. The program module 310 may include a kernel 320, middleware 330, an API 360, and/or applications 370. At least some of the program module 310 may be preloaded on an electronic device, or may be downloaded from an external electronic device (e.g., the electronic device 102 or 104, or the server 106).

The kernel 320 (e.g., the kernel 141) may include, for example, a system resource manager 321 and/or a device driver 323. The system resource manager 321 may control, allocate, or collect system resources. According to an embodiment of the present disclosure, the system resource manager 321 may include a process management unit, a memory management unit, a file system management unit, and the like. The device driver 323 may include, for example, a display driver, a camera driver, a Bluetooth driver, a shared memory driver, a USB driver, a keypad driver, a Wi-Fi driver, an audio driver, or an Inter-Process Communication (IPC) driver. For example, the middleware 330 may provide a function required in common by the applications 370, or may provide various functions to the applications 370 through the API 360 so as to enable the applications 370 to efficiently use the limited system resources in the electronic device. According to an example embodiment of the present disclosure, the middleware 330 (e.g., the middleware 143) may include at least one of a run time library 335, an application manager 341, a window manager 342, a multimedia manager 343, a resource manager 344, a power manager 345, a database manager 346, a package manager 347, a connectivity manager 348, a notification manager 349, a location manager 350, a graphic manager 351, and a security manager 352.

The runtime library 335 may include a library module that a compiler uses in order to add a new function through a programming language while an application 370 is being executed. The runtime library 335 may perform input/output management, memory management, the functionality for an arithmetic function, or the like. The application manager 341 may manage, for example, a life cycle of at least one of the applications 370. The window manager 342 may manage Graphical User Interface (GUI) resources used by a screen. The multimedia manager 343 may recognize a format required for reproduction of various media files, and may perform encoding or decoding of a media file by using a codec suitable for the corresponding format. The resource manager 344 may manage resources of a source code, a memory, and a storage space of at least one of the applications 370. The power manager 345 may operate together with, for example, a Basic Input/Output System (BIOS) or the like to manage a battery or power source and may provide power information or the like required for the operations of the electronic device. The database manager 346 may generate, search for, and/or change a database to be used by at least one of the applications 370. The package manager 347 may manage installation or an update of an application distributed in a form of a package file.

For example, the connectivity manager 348 may manage wireless connectivity such as Wi-Fi or Bluetooth. The notification manager 349 may display or notify of an event such as an arrival message, promise, proximity notification, and the like in such a way that does not disturb a user. The location manager 350 may manage location information of an electronic device. The graphic manager 351 may manage a graphic effect which will be provided to a user, or a user interface related to the graphic effect. The security manager 352 may provide all security functions required for system security, user authentication, or the like. According to an embodiment of the present disclosure, when the electronic device (e.g., the electronic device 101) has a telephone call function, the middleware 330 may further include a telephony manager for managing a voice call function or a video call function of the electronic device. The middleware 330 may include a middleware module that forms a combination of various functions of the above-described components. The middleware 330 may provide a module specialized for each type of OS in order to provide a differentiated function. Further, the middleware 330 may dynamically remove some of the existing components or add new components. The API 360 (e.g., the API 145) is, for example, a set of API programming functions, and may be provided with a different configuration according to an OS. For example, in the case of Android™ or iOS™, one API set may be provided for each platform. In the case of Tizen™, two or more API sets may be provided for each platform.

The application 370 may, for example, include a home 371, a dialer 372, a short message service (SMS)/multimedia message service (MMS) 373, an instant message (IM) 374, a browser 375, a camera 376, an alarm 377, a contact 378, a voice dial 379, an electronic mail (e-mail) 380, a calendar 381, a media player 382, an album 383, a watch 384, a health care (e.g., measuring a momentum, a blood sugar or the like), or an environment information (e.g., air pressure, humidity, or temperature information) provision application. According to an embodiment, the application 370 may include an information exchange application capable of supporting information exchange between an electronic device and an external electronic device. The information exchange application may, for example, include a notification relay application for relaying specific information to the external electronic device, or a device management application for managing the external electronic device. For example, the notification relay application may relay notification information provided in another application of the electronic device, to the external electronic device, or receive notification information from the external electronic device and provide the received notification information to a user. The device management application may, for example, install, delete, or refine a function (e.g., turned-on/turned-off of the external electronic device itself (or some components) or adjustment of a brightness (or resolution) of a display) of the external electronic device which communicates with the electronic device, or an application which operates in the external electronic device. According to an embodiment, the application 370 may include an application (e.g., a health care application of a mobile medical instrument) designated according to properties of the external electronic device. According to an embodiment, the application 370 may include an application received from the external electronic device. At least a part of the program module 310 may be implemented (e.g., executed) as software, firmware, hardware (e.g., the processor 210), or a combination of at least two or more of them, and may include a module for performing one or more functions, a program, a routine, sets of instructions or a process.

The term “module” used in the document may include a unit consisting of hardware, software or firmware, and may be, for example, used interchangeably with the term “logic”, “logic block”, “component”, “circuitry” or the like. The “module” may be an integrally configured component or the minimum unit performing one or more functions or a part thereof. The “module” may be implemented mechanically or electronically, and may, for example, include an application-specific integrated circuit (ASIC) chip, a field-programmable gate array (FPGA) or a programmable logic device, which has been known or will be developed in future, performing some operations. At least a part of an apparatus (e.g., modules or functions thereof) or method (e.g., operations) according to various embodiments may, for example, be implemented as an instruction which is stored in a computer-readable storage medium (e.g., the memory 130) in the form of a program module. In response to the instruction being executed by a processor (e.g., the processor 120 of FIG. 1 or the processor 210 of FIG. 2), the processor may perform a function corresponding to the instruction. The computer-readable recording medium may include a hard disk, a floppy disk, a magnetic medium (e.g., a magnetic tape), an optical recording medium (e.g., a compact disk-read only memory (CD-ROM), a digital versatile disk (DVD)), a magneto-optical medium (e.g., a floptical disk), an internal memory, etc. The instruction may include a code which is made by a compiler or a code which is executable by an interpreter. The module or program module according to various embodiments may include at least one or more of the aforementioned constituent elements, or omit some of them, or further include another constituent element. Operations carried out by the module, the program module or the another constituent element according to various embodiments may be executed in a sequential, parallel, repeated or heuristic manner, or at least some operations may be executed in different order or may be omitted, or another operation may be added.

An electronic device according to various embodiments of the disclosure may include: a communication module; a sensor module; a processor configured to be electrically connected to the communication module and the sensor module; and a memory configured to be electrically connected to the processor, wherein the memory is controlled to store, when executed, instructions that cause the processor to receive a first healthcare program from at least one another electronic device using the communication module, to generate a first schedule including at least one activity based on the received first healthcare program, to provide a notification to a user based on the first schedule, to sense an activity of the user related to a first activity during the at least one activity, using the sensor module, to link, with the first activity, a first item included in an exercise application associated with the first activity, and to reflect, in the first item, activity information of the user related to the first activity acquired using the sensor module.

According to an embodiment, the instructions may cause the processor to detect at least one of first healthcare program information, session information, and activity information based on the received first healthcare program.

According to an embodiment, the instructions may cause the processor to generate the first schedule based on the session information and the activity information.

According to an embodiment, the instructions may cause the processor to receive a second healthcare program from at least one server using the communication module, to generate a second schedule including at least one activity based on the received second healthcare program, to provide a notification to the user based on the second schedule, to sense an activity of the user related to a second activity during the at least one activity, using the sensor module, and to link the second activity with the first item when the second activity overlaps the first activity.

According to an embodiment, the instructions may cause the memory to store first healthcare program progress information including a first healthcare program progress status according to the user activity information and the first schedule.

According to an embodiment, the instructions may cause the processor to detect feedback of the user associated with the first healthcare program, and to transmit at least one of the first healthcare program progress information and the feedback of the user to the other electronic device.

According to an embodiment, the instructions may cause the processor to receive a service generated based on the at least one of the first healthcare program progress information and the feedback of the user from the other electronic device.

According to an embodiment, the instructions may cause the processor to determine whether an item corresponding to the first activity exists in the exercise application, to generate an item corresponding to the activity when the item corresponding to the first activity does not exist in the exercise application, and to link the generated item with the first activity.

According to an embodiment, the instructions may cause the processor to determine whether the first healthcare program is completed based on the activity information of the user reflected in the first item of the exercise application.

An electronic device according to various embodiments of the disclosure may include a sensor configured to be functionally connected to the electronic device; and a processor, wherein the processor may receive an input of healthcare program information, may determine whether an input of session information is detected, may determine whether an activity of a user is detected when the input of the session information is not detected, to generate the session information based on the activity of the user when the activity of the user is detected, and to generate a healthcare program based on the healthcare program information and the session information.

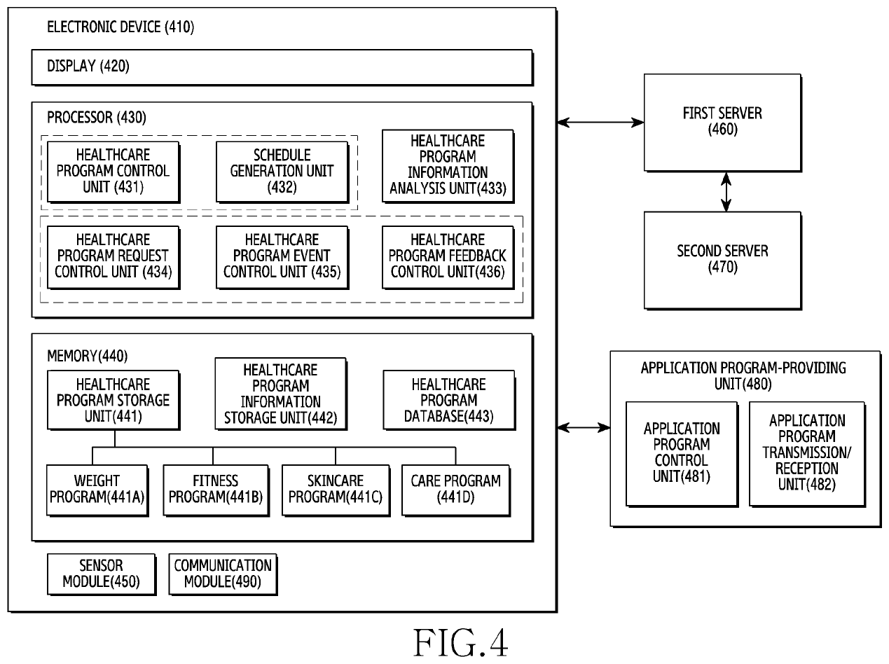

FIG. 4 is a block diagram illustrating the configuration of an electronic device and a server according to various embodiments of the disclosure.

Referring to FIG. 4, in an embodiment, an electronic device 410 may include a display 420, a processor 430, a memory 440, a sensor module 450, and a communication module 490. The electronic device according to various embodiments of the disclosure may include at least one of, for example, a smartphone, a tablet personal computer (PC), a mobile phone, a video phone, an e-book reader, a desktop PC, a laptop PC, a netbook computer, a workstation, a server, a personal digital assistant (PDA), a portable multimedia player (PMP), a MP3 player, a mobile medical device, a camera, and a wearable device. According to various embodiments, the wearable device may include at least one of an accessory-type device (e.g., a watch, a ring, a bracelet, a necklace, a pair of glasses, a shoe, a contact lens, or a head-mounted device (HIVID)), a fabric- or garment-integrated device (e.g., an electronic garment), a body-attachment-type device (e.g., a skin pad or tattoo), and a bio-implantable device (e.g., an implantable circuit). However, the wearable device is not limited thereto.

In an embodiment, the display 420 may display at least one healthcare program. The healthcare program may be a service to which a user of the electronic device 410 subscribes. For example, the healthcare program may be a sort of schedule that suggests that a user performs a predetermined activity for a predetermined time period. The healthcare program may be provided to the user through a separate user interface and may be provided to the user through an exercise application. In this specification, for convenience of description, the case in which at least one healthcare program and an exercise application interwork with each other will be described. The healthcare program may be generated by a provider and updated on a first server 460, and the user of the electronic device 410 may receive the healthcare program through the first server 460. In addition, the user of the electronic device 410 may directly generate a healthcare program and may supply the healthcare program to the first server 460. However, the disclosure is not limited thereto. Hereinafter, for convenience of description, a person who executes the healthcare program is referred to as a user and a person who generates the healthcare program is referred to as a provider.

In an embodiment, the healthcare program may include a weight program 441a, a fitness program 441b, a skincare program 441c, and a care program 441d. For example, the weight program 441a may include a schedule for losing 5 kg in two weeks, the fitness program 441b may include a schedule for making a bikini body, the skincare program 441c may include a schedule for skin improvement, and the care program 441d may include a schedule for managing constipation through water ingestion. However, the healthcare program is not limited thereto, and the healthcare program may include various healthcare programs related to the health of the user of the electronic device 410, and the like.

In an embodiment, the processor 430 may generate and manage at least one healthcare program, may control the communication module 490 to transmit or receive the at least one healthcare program to or from another electronic device, and may perform control to store healthcare program-related information in the memory 440. For example, the processor 430 may include a healthcare program control unit 431, a schedule generation unit 432, a healthcare program information analysis unit 433, a healthcare program request control unit 434, a healthcare program event control unit 435, and a healthcare program feedback control unit 436. However, the processor 430 is not limited thereto, and may include other configurations necessary for healthcare program management. Alternatively, the processor 430 may include only the healthcare program control unit 431, and the healthcare program control unit 431 may control all operations (acting as the schedule generation unit, the healthcare program information analysis unit, the healthcare program request control unit, the healthcare program event control unit, and the healthcare program feedback control unit).

In an embodiment, the healthcare program control unit 431 may perform control to register the healthcare program, to cancel the healthcare program, to subscribe to the healthcare program, to interrupt the healthcare program, to manage a healthcare program progress, to generate a healthcare program-related event, to provide a recommended healthcare program list, and the like. However, the healthcare program control unit 431 is not limited thereto, and may control various functions so that a user can use the healthcare program easily.

In an embodiment, the healthcare program control unit 431 may manage healthcare program-related information. The healthcare program-related information may include healthcare program information, session information, activity information, and schedule information. The healthcare program information may include the title, description, duration, organization, expert information, etc., of the healthcare program. The session information may include at least one piece of activity information, and may include metadata for schedule generation. For example, the metadata for schedule generation may include a span, a frequency, repetition days during the span, repetition counts, and the like. The activity information may include activities of the smallest unit that the user has to perform, and the activities may be linked one-to-one with the items of the exercise application. For example, the activity could be drinking eight glasses of water or squatting one minute, etc. The schedule information may include a schedule including a date and activity information to be performed by the user by combining the session information and the activity information. However, the healthcare program-related information is not limited thereto, and may be variously configured in order to improve the health of the user.

Meanwhile, according to an embodiment of the disclosure, each healthcare program may include partially overlapping information. For example, the healthcare program may include a first healthcare program and a second healthcare program. A first schedule including a first activity may be generated based on the first healthcare program, and a second schedule including a second activity may be generated based on the second healthcare program. In this case, the first activity and the second activity may overlap each other. For example, a weight program may include an activity of drinking eight glasses of water, and a skincare program may also include the same activity of drinking eight glasses of water. In this case, the overlapping activities can be linked with the same item in the exercise application.

In an embodiment, the schedule generation unit 432 may generate schedule information to be performed by the user based on the healthcare program information, the session information, and the activity information. Meanwhile, the healthcare program-related information may be extracted by the processor 430 or by the schedule generation unit 432 based on at least one healthcare program received through the communication module 490. However, the disclosure is not limited thereto.

In an embodiment, the healthcare program information analysis unit 433 may monitor the activity information of the user based on at least one piece of sensor data acquired by the sensor module 450, may analyze whether the user performs the healthcare program according to the schedule included in the schedule information, and may manage the status of the healthcare program. The status of the healthcare program may include the version of the healthcare program, the progress of the healthcare program according to the activity information of the user, and the like. For example, the healthcare program information analysis unit 433 may determine whether the activity of the user according to the schedule of the healthcare program is completed, whether the user has performed an activity scheduled for that day, and the like. In addition, the healthcare program information analysis unit 433 may generate healthcare program progress information including the result of determining whether the activity of the user has been completed or the like. However, the disclosure is not limited thereto.

In an embodiment, the healthcare program request control unit 434 may request the healthcare program from the first server 460 through the communication module 490, and may receive the healthcare program from the first server 460 via the communication module 490. For example, if a user is interested in weight loss, the healthcare program request control unit 434 may request a healthcare program list related to weight loss from the first server 460 via the communication module 490, and may receive the healthcare program list via the communication module 490 in accordance with the request. For example, the healthcare program request control unit 434 may receive information about the location of a representative image of the healthcare program in the form of a URI via the communication module 490, and may download the received representative image from the first server 460 via the communication module 490 to generate a bitmap object. The healthcare program request control unit 434 may transmit healthcare program progress information generated by the healthcare program information analysis unit 433 to the first server 460 via the communication module 490. However, the disclosure is not limited thereto.

In an embodiment, the healthcare program event control unit 435 may receive or generate a healthcare program-related event from the first server 460 via the communication module 490. For example, if a weight-loss healthcare program achieves a target weight loss in the middle of a schedule, the healthcare program-related event may be a congratulatory message or a message advertising a product that can assist in achieving a healthcare program goal. However, the disclosure is not limited thereto. Meanwhile, the healthcare program event control unit 435 may transmit the healthcare program-related event to the second server 470 using the communication module 490, and may receive the healthcare program-related event from the second server 470 using the communication module 490.

In an embodiment, the healthcare program feedback control unit 436 may transmit user feedback on the healthcare program to the first server 460 using the communication module 490. For example, if the entire healthcare program is completed or one activity is completed during a schedule, the healthcare program feedback control unit 436 may display a notification via the display 420 to provide the user with an opportunity to input the feedback, may store the feedback input by the user in the memory 440, and may transmit the stored feedback in real time to the first server 460 via the communication module 490 or send the same all at once after the healthcare program is completed. In addition, the healthcare program feedback control unit may transmit a user's question related to the healthcare program to the first server 460 via the communication module 490, and may receive a response of an expert or a healthcare program provider to the corresponding question from the first server 460 via the communication module 490. However, the disclosure is not limited thereto. Meanwhile, the healthcare program feedback control unit 436 may transmit the user feedback on the healthcare program to the second server 470 using the communication module 490, and may receive the user feedback from the second server 470 using the communication module 490.

In an embodiment, the memory 440 may include a healthcare program storage unit 441, a healthcare program information storage unit 442, and a healthcare program database 443. The healthcare program storage unit 441 may store content of the healthcare program in the memory 440 and may store cache information for improving query performance in the memory 440. For example, the healthcare program storage unit 441 may store the healthcare programs such as a weight program 441a, a fitness program 441b, a skincare program 441c, and a care program 441d in the memory 440. The content of the healthcare program may include static information and resource information such as healthcare program titles, descriptions, and categories. The healthcare program information storage unit 442 may store the healthcare program information, the session information, the activity information, schedule information, and the like, which are extracted from the healthcare program, in the memory 440, and may store user activity information and healthcare program progress information according to user performance in the memory 440. The user activity information may be information corresponding to each activity execution result included in the schedule, and the healthcare program progress information may include a healthcare program progress result based on the user activity information. The healthcare program database 443 may be a database for storing a healthcare program log. However, the disclosure is not limited thereto.

In an embodiment, the sensor module 450 may detect activity information of the user via at least one sensor. For example, the sensor may include a nine-axis sensor, a barometer, a heart rate sensor, a pressure sensor, and the like. For example, the nine-axis sensor may be referred to as a motion sensor, and may include at least one of an acceleration sensor, a gyroscope, and a geomagnetic sensor. The activity information of the user according to various embodiments may include changes in body movements (e.g., motion, position movement, etc.) and biometric information (e.g., heart rate, oxygen saturation, consumed calories, etc.), or the location of the user.

According to an embodiment of the disclosure, the electronic device 410 may collect the activity information of the user through at least one sensor module 450 provided in the electronic device 410. For example, a smart watch, a smart phone, a smart headset, and a smart bracelet may include a nine-axis sensor, a barometer, and a heart rate sensor. Smart glasses may include a 9-axis sensor and a barometer. Smart shoes may include a 9-axis sensor, a barometer, and a pressure sensor. However, the disclosure is not limited thereto.

According to an embodiment of the disclosure, the electronic device 410 may collect the activity information of the user using at least one peripheral device. For example, the activity information of the user that can be recognized may be different depending on the position where the electronic device 410 is attached to or is in contact with the user's body, or the type of the sensor included in the electronic device 410. Accordingly, the electronic device 410 may detect the activity information of the user in conjunction with at least one peripheral device so as to correspond to the exercise type of the user and sensor information of the electronic device 410. For example, the electronic device 410 may be connected to at least one peripheral device selected from among a smart headset, a smart bracelet, smart glasses, smart shoes, and a fitness device via the communication module 490. The electronic device 410 may be linked with at least one peripheral device which is connected for communication, thereby collecting the user activity information. For example, the electronic device 410 may be connected to the peripheral device via a local area communication method such as Wi-Fi, Bluetooth, Bluetooth low energy (BLE), near-field communication (NFC), and the like. However, the disclosure is not limited thereto.

In an embodiment, the communication module 490 may connect the first server 460 and the electronic device 410. For example, the communication module 490 may include a cellular module, a Wi-Fi module, a BT module, a global navigation satellite system (GNSS) module, an NFC module, and the like. The electronic device 410 may receive the healthcare program from the first server 460 via the communication module 490. However, the disclosure is not limited thereto.

In an embodiment, the first server 460 may provide the healthcare program to the electronic device 410 via the communication module 490. The first server 460 may provide all functions for healthcare program registration, management, etc., associated with the healthcare program control unit 431 of the electronic device 410 to the electronic device 410 via the communication module 490. The first server 460 may be provided with a user profile including the user's age and gender, user behavior information such as browsing, searching, or downloading the healthcare program, user feedback, and the like from the electronic device 410 via the communication module 490. The first server 460 may provide a recommended healthcare program list to the user of the electronic device 410 via the communication module 490 based on the user profile, the user behavior information, the user feedback, and context information including the season, the current time, and the like. However, the disclosure is not limited thereto.

In an embodiment, the first server 460 may provide a user interface for producing the healthcare program to the healthcare program provider via the communication module 490. In addition, the first server 460 may provide a user interface for confirming statistics for each user and each healthcare program to the healthcare program provider via the communication module 490, and may provide a user interface for confirming a user's message and feedback related to the healthcare program via the communication module 490. The first server 460 may receive a message such as expert advice from the healthcare program provider and may forward the received message to the electronic device 410 via the communication module 490, and may provide a user interface through which the healthcare program provider can directly transmit a message to the user, via the communication module 490. However, the disclosure is not limited thereto.

Meanwhile, according to an embodiment of the disclosure, the user of the electronic device 410 may be a provider that generates the healthcare program through the user interface provided by the first server 460 while receiving the healthcare program through the communication module 490 from the first server 460. That is, as the first server 460 provides the user interface for producing the healthcare program via the communication module 490, the user of the electronic device 410 may be provided with an opportunity to act as a provider capable of easily producing and distributing the healthcare program. Accordingly, the electronic device 410 may be a device used by the user who executes the healthcare program or a device used by the healthcare program provider. However, the disclosure is not limited thereto.

In an embodiment, the second server 470 may be a server operated by a specialized provider that produces a plurality of healthcare programs, and may provide the plurality of healthcare programs to the first server 460. However, the disclosure is not limited thereto.

In an embodiment, the healthcare program provider may provide the healthcare program via an application program-providing unit 480 without a server. The application program-providing unit 480 may be an external electronic device capable of providing an application program to the user through the communication module 490. For example, if the user of the electronic device 410 downloads an application program, a plurality of healthcare programs may be included in the application program. The application program-providing unit 480 may include an application program control unit 481 and an application program transmission/reception unit 482. The application program control unit 481 may allow a separate healthcare program to be included in the application program, and the application program transmission/reception unit 482 may transmit the application program including the healthcare program to the electronic device 410 through the communication module 490. Meanwhile, unlike this, the healthcare program may be provided through the application program stored previously in the electronic device 410 via the communication module 490. In this case, the previously stored application program may be an application program that can provide health-related information of the user or can manage the user's health. However, the disclosure is not limited thereto.

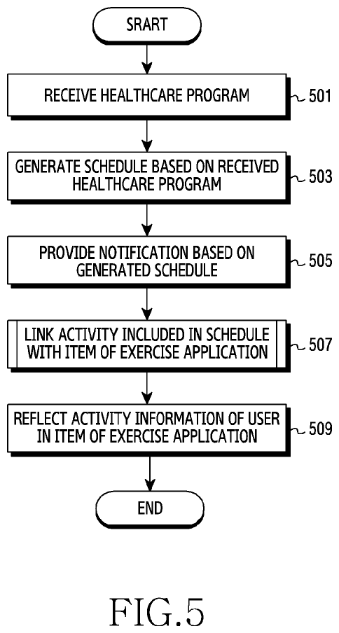

FIG. 5 is a flowchart illustrating an operation method of an electronic device according to various embodiments of the disclosure.

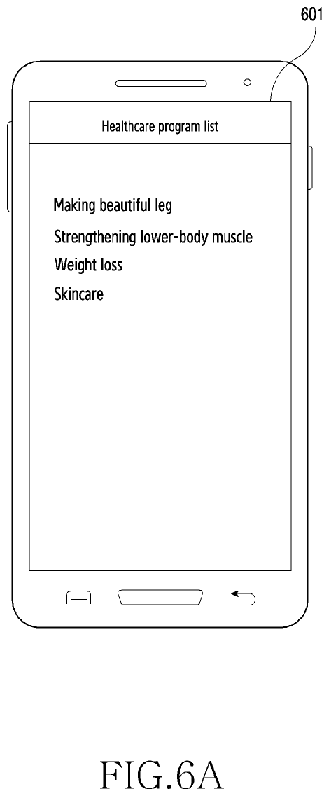

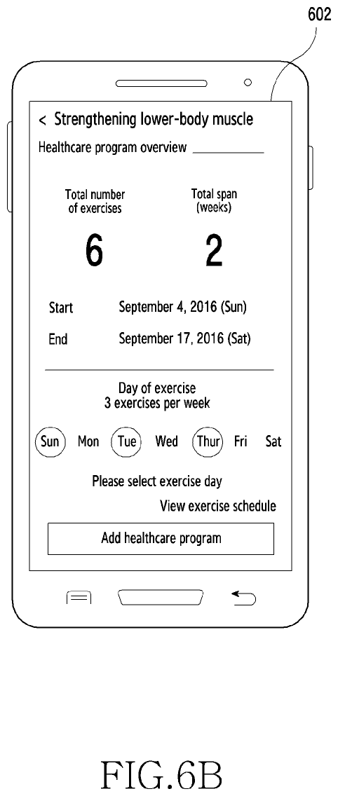

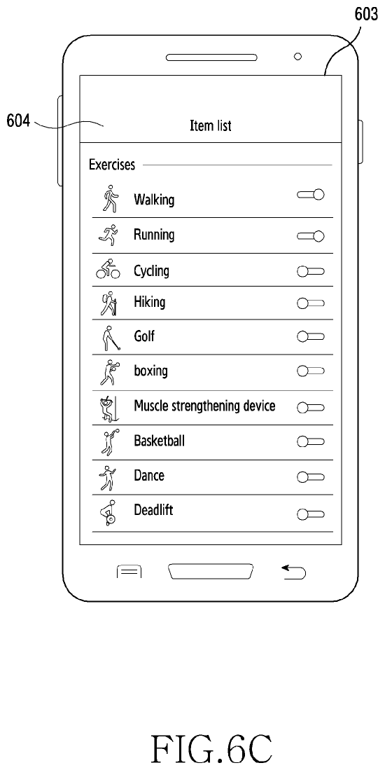

FIGS. 6A to 6C are exemplary diagrams illustrating an exercise application interworking with a healthcare program according to an embodiment of the disclosure.

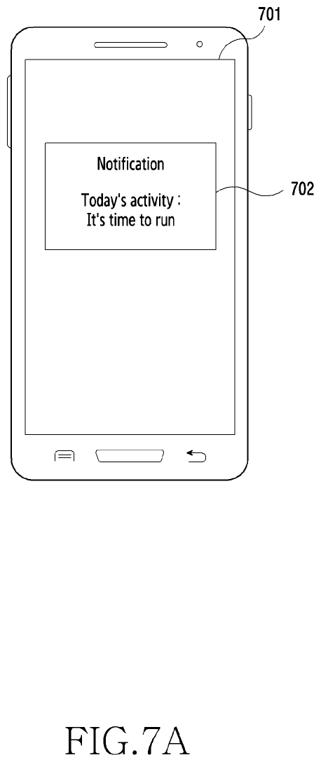

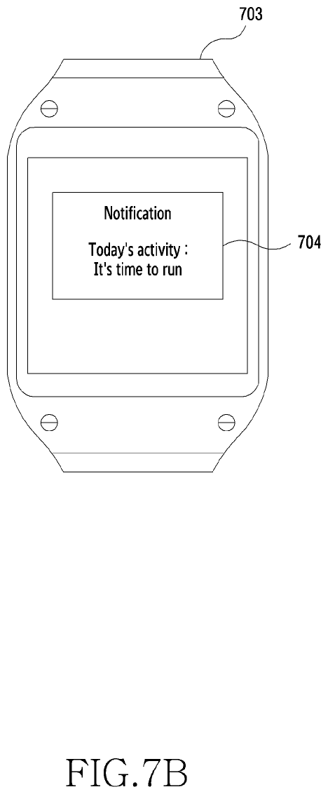

FIGS. 7A and 7B are exemplary diagrams illustrating a method of displaying an alarm according to an embodiment of the disclosure.

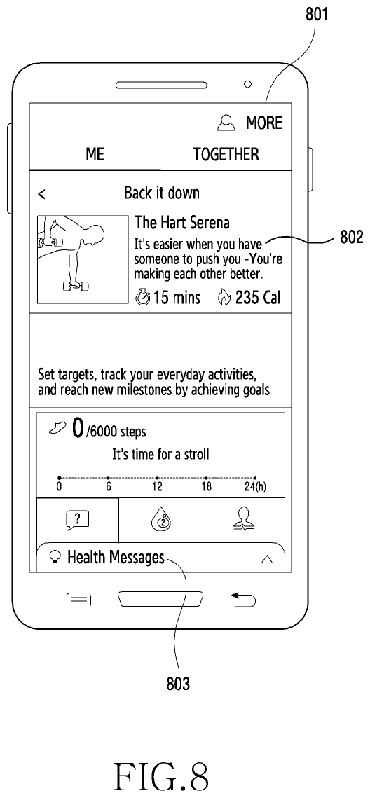

FIG. 8 is an exemplary diagram illustrating a method of displaying a healthcare program according to an embodiment of the disclosure.

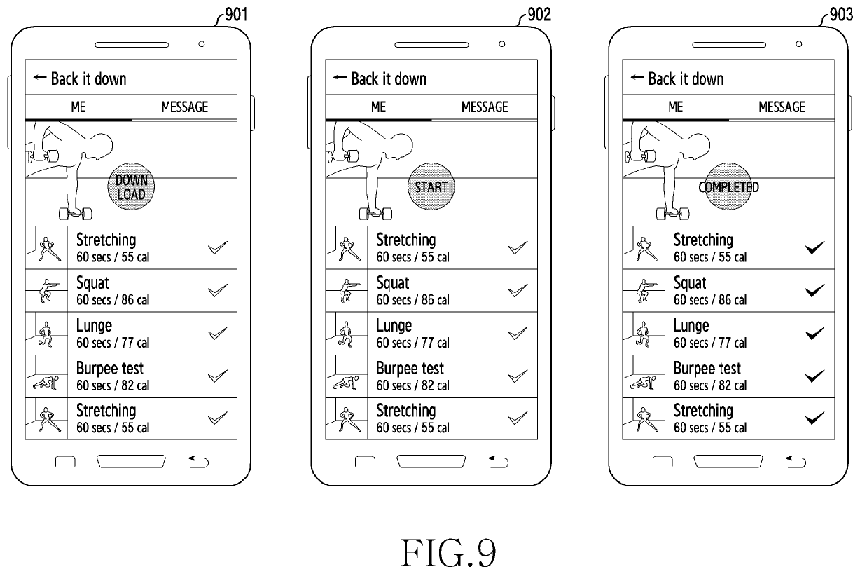

FIG. 9 is an exemplary diagram illustrating a method of performing a healthcare program according to an embodiment of the disclosure.

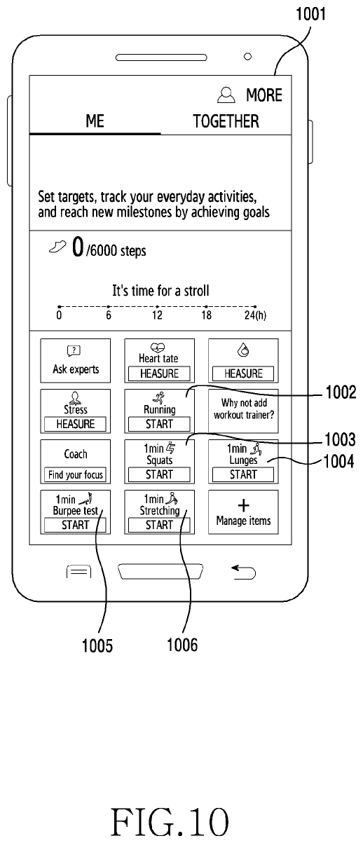

FIG. 10 is an exemplary diagram illustrating a method of confirming a healthcare program progress result according to an embodiment of the disclosure.

Referring to FIG. 5, in operation 501, in an embodiment, the processor 430 (shown in FIG. 4) of the electronic device 410 (shown in FIG. 4) may receive at least one healthcare program from another electronic device via the communication module 490 (shown in FIG. 4). The other electronic device may be an electronic device of a provider that directly generates a program or a server. For convenience of description, an operation in which the electronic device 410 receives the healthcare program from the first server 460 via the communication module 490 will be described as an example. The healthcare program may be a service to which the user of the electronic device 410 subscribes via the communication module 490. For example, the healthcare program may be a sort of schedule that suggests that a user performs a predetermined activity for a predetermined time period. However, the disclosure is not limited thereto.

Referring to FIG. 6A, in an embodiment, the electronic device may display a healthcare program received through the display 420 (shown in FIG. 4) on an exercise application. For example, the processor 430 may display a healthcare program list received from a management page 1001 of the exercise application through the display 420. The healthcare program list may include, for example, a beautiful-leg-making healthcare program, a lower-body muscle-strengthening healthcare program, a weight-loss healthcare program, a skincare healthcare program, and the like. As described above, the received healthcare program may be provided in a separate user interface rather than through the exercise application. However, the disclosure is not limited thereto.

In operation 503, in an embodiment, the processor 430 may generate a schedule including at least one activity based on the healthcare program received through the communication module 490. For example, the processor 430 may extract healthcare program information, session information, and activity information from the healthcare program received through the communication module 490, and may generate schedule information based on the extracted healthcare program information, session information, and activity information. The schedule information may include at least one schedule, and the schedule may refer to an activity plan of the user who is to perform the healthcare program. The session information may include metadata for schedule generation as described above, and the metadata for schedule generation may include a span, a frequency, repetition days during the span, repetition counts, and the like. Specifically, the processor 430 may display a title, a description, and the like of the program through the display 420 based on the healthcare program information extracted from the healthcare program. In addition, the processor 430 may determine a healthcare program start date and a healthcare program end date based on the session information extracted from the healthcare program received through the communication module 490. For example, if an exercise span extracted from the session information is 14 days and today's date is September 4, the start date may be determined to be September 4, and the end date may be determined to be September 17. In addition, if a frequency extracted from the session information is 7 days and extracted repetition dates are 1/3/5, the processor 430 may determine a schedule of a 7-day unit frequency, and may generate an activity schedule on day 1, day 3, and day 5 for 7 days. In this case, the processor 430 may generate an activity schedule on September 4th/September 6th/September 8th/September 11th/September 13th/S eptemb er 15th. In addition, the processor 430 may determine activities to be performed in each activity schedule based on the activity information extracted from the received healthcare program. For example, the activity information may include stretching, squatting, lunge, and burpee test. The processor 430 may determine a stretching activity on a first activity date based on the extracted activity information, may determine a squat activity on a second activity date, may determine a lunge activity on a third activity date, may determine a burpee test activity on a fourth activity date, may determine the stretching activity again on a fifth activity date, and may repeat these activities in sequence. However, the disclosure is not limited thereto.

Referring to FIG. 6B, in an embodiment, the processor 430 may display the generated schedule of the healthcare program on a healthcare program detail page 602 of the exercise application via the display 420. As described above, the lower-body muscle-strengthening healthcare program may be displayed on the healthcare program detail page 602 via the display 420. In the healthcare program overview, a total number of exercise sessions of 6 times, a total span of 2 weeks, the start date on September 4, the end date on September 17, and the exercise days of Sunday/Tuesday/Thursday may be displayed. However, the disclosure is not limited thereto, and healthcare program details may be displayed in various ways to facilitate understanding of the healthcare program.

Meanwhile, referring to FIG. 6C, in an embodiment, the processor 430 may set a plurality of items in an item page 603 of the exercise application. Items displayed on the item page 603 may be functions provided in the exercise application of the electronic device 410 in advance regardless of whether the healthcare program is received or not. User activities corresponding to each of the plurality of items may be detected in real time through the sensor module 450, and the detected user activities may be stored. However, the disclosure is not limited thereto.

In operation 505, in an embodiment, the processor 430 may provide a notification using the display 420, an audio module, or a motor based on the generated schedule. That is, the processor 430 may display the notification on the display 420 of the electronic device 410 and may provide the notification to the user using voice, vibration, or the like. Alternatively, if there is a peripheral device connected through the communication module, the processor 430 may provide a notification via the peripheral device. For example, in the case of a healthcare program in which a fitness healthcare program is performed three times a week for four weeks, the processor 430 may display, on the display 420, a notification that it is time to perform activities included in the healthcare program at specific times every Monday, Wednesday, and Friday, may provide a notification that it is time to perform the activities by voice through the audio module, and may provide a notification that it is time to perform the activities by vibration through the motor. In an embodiment, if the healthcare program does not include the specific time at which the activity is to be performed, the processor 430 may provide a notification via the display 420 at a time that the user can perform the activity based on context information. The context information may include calendar information, user schedule information, SNS information, SMS information, weather information, holiday information, and the like. In an embodiment, if the healthcare program includes a specific time at which the activity is to be performed, the processor 430 may provide a notification prior to the specific time in order to enable preparation for the activity. For example, if the specific time at which the activity is to be performed is 14:00, the processor 430 may provide a first alarm at 12:00 and a second alarm at 13:00. The duration and frequency of the alarm may be changed in various ways.

Referring to FIG. 7A, the processor 430 may display, on a display screen 701, a notification 702 indicating “Today's activity: It's time to run” through the display 420. In addition, referring to FIG. 7B, even when the electronic device 410 is a wearable device, a notification 704 indicating “It's time to run” may be displayed on the display screen 703. In addition, for example, the processor 430 may extract the frequency, the repetition days, and the like included in the session information, and may provide a notification indicating that the user should take medication on every Monday, Wednesday, and Friday at 8:00 am, 1:00 pm, and 6:00 pm. In addition, for example, in the case where the healthcare program does not include the specific time, the processor 430 may provide a notification when the display 420 is turned on or when an unlock event of the display 420 is detected.

In operation 507, in an embodiment, the processor 430 may link at least one activity included in the schedule with at least one item included in the exercise application. In particular, when the activity of the user associated with the at least one activity included in the schedule is detected, the processor 430 may link the at least one activity included in the schedule with the item of the exercise application. Referring to FIG. 6C, the exercise application may include a plurality of items corresponding to various activities that the user can perform, and may display an item list in a management page 603. For example, the item list of the exercise application may include items such as walking, running, cycling, hiking, golf, boxing, use of a muscle strengthening device, basketball, dancing, deadlifting, and the like. The exercise application may manage each item in item management. The activity information of the user sensed by the sensor module 450 of the electronic device 410 may be linked with the corresponding item of the exercise application. For example, when the activity included in the healthcare program is a running for 20 minutes, the activity information of the user who sensed the execution of the user's running may be linked with the running item of the exercise application. However, the disclosure is not limited thereto.

Meanwhile, according to an embodiment of the disclosure, a time when the activities included in the schedule are linked with the items of the exercise application may be realized simultaneously with a time when the healthcare program is received to extract the healthcare program-related information. That is, the activities included in the healthcare program may be linked with the items of the exercise application, regardless of whether the activity of the user corresponding to the activity included in the schedule is detected. However, the disclosure is not limited thereto.

Meanwhile, according to an embodiment, the processor 430 may also link activities overlapping the plurality of healthcare programs with the items of the exercise application. In an embodiment, the healthcare program may include a first healthcare program and a second healthcare program. A first schedule including a first activity may be generated based on the first healthcare program, and a second schedule including a second activity may be generated based on the second healthcare program. In this case, the first activity and the second activity may overlap each other. For example, a healthcare program to which a user subscribes may be a weight program and a skincare program. The weight management healthcare program may include drinking 8 glasses of water daily, and the skincare healthcare program may also include drinking 8 glasses of water daily. In this case, the processor 430 may link the drinking 8 glasses of water daily of the weight management healthcare program and the drinking 8 glasses of water daily of the skincare healthcare program which overlaps each other together with a water (water-drinking) item of the exercise application. That is, the items of the exercise application may be linked with each activity unit included in the healthcare program, rather than the healthcare program unit. However, the disclosure is not limited thereto.

In operation 509, in an embodiment, the processor 430 may reflect the activity information of the user in the item of the exercise application, and may perform control to store instructions that cause the activity information of the user to be reflected in the item in the memory 440. For example, after detecting the activity information of the user who performs a running activity included in the schedule using the sensor module 450, the processor 430 may control the activity information of the user to be stored in a running item of the exercise application. Therefore, the activity information of the user may be stored in the item of the exercise application without separate input by the user for the activity result, and the user may confirm the activity result by clicking on the item of the exercise application. In addition, in an embodiment, the processor 430 may detect the activity information of the user using an audio module or a camera module. Unlike this, the processor 430 may receive the activity information from the user through the audio module or the camera module, and the received activity information may be reflected in the item of the exercise application. Also, in an embodiment, the processor 430 may receive the activity information via a touch input of the user using an input device such as a touch panel. However, the disclosure is not limited thereto.

Referring to FIG. 8, in an embodiment, the processor 430 may display the healthcare program received via the display 420 on a main page 801 of the exercise application. The processor 430 may display the schedule extracted from the healthcare program through the display 420 on the main page 801 of the exercise application. The processor 430 may display a summary of the day's tasks on a dashboard 802 of the exercise application every day that there is an activity according to the schedule via the display 420. In addition, the processor 430 may display a health message 803, such as advice on what to do that day, via the display 420. However, the disclosure is not limited thereto.

Referring to FIG. 9, in an embodiment, the processor 430 may display the details of the activity included in the schedule via the display 420. For example, the processor 430 may display a moving image download page 901 related to the activity in the exercise application via the display 420 when there is a moving image related to the activity, and may receive the moving image related to the activity from the first server 460 via the communication module 490 based on a user input. The processor 430 may display stretching, squat, lunge, burpee test, and stretching included in today's activities in sequence on an execution page 902 of the exercise application through the display 420, and may display the result of the today's activities on a completion page 903 of the exercise application through the display 420 when the activity of the user is detected through the sensor module 450. However, the disclosure is not limited thereto.

Referring to FIG. 10, in an embodiment, the processor 430 may display the activity result of the user on an item log 1001 of the exercise application via the display 420. For example, the processor 430 may display a running log 1002, a squat log 1003, a lunge log 1004, a burpee test log 1005, and a stretching log 1006 on the item log 1001 of the exercise application via the display 420, and the user may confirm the activity result through the above-mentioned logs. However, the disclosure is not limited thereto, and the user can confirm the activity result in each item in the item management shown in FIG. 6C.

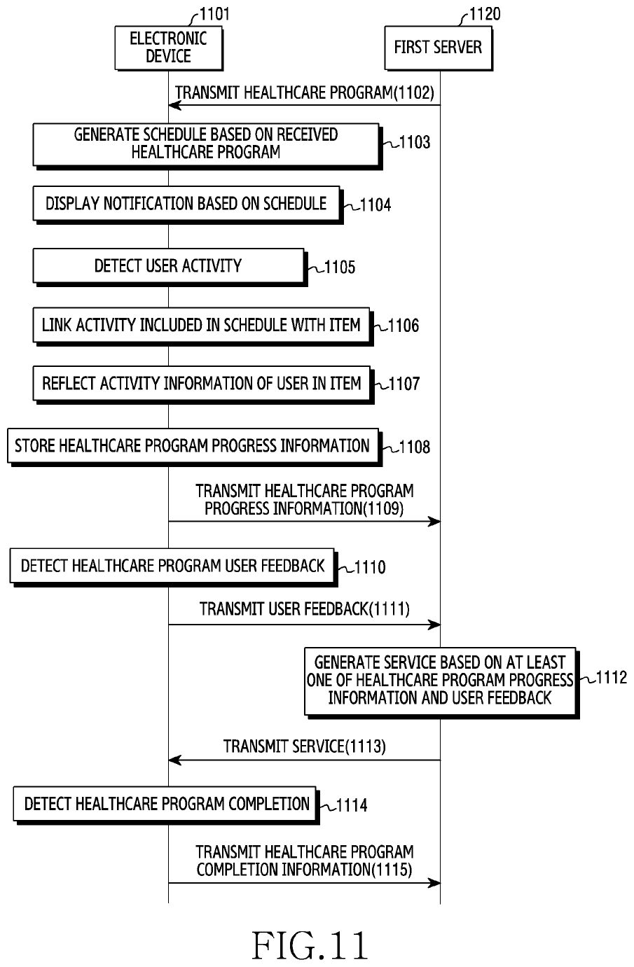

FIG. 11 is a flowchart illustrating a system between an electronic device according to an embodiment of the disclosure and a server.

Referring to FIG. 11, an electronic device 1101 may communicate with a first server 1120 using the communication module 490 (shown in FIG. 4). In operation 1102, in an embodiment, the first server 1120 may transmit a healthcare program to the electronic device 1101 via the communication module 490. For example, the electronic device 1101 may provide a healthcare program list received from the first server 1120 through the communication module 490, to a user through the display 420. Upon receiving an input of selecting at least one healthcare program from the healthcare program list from the electronic device 1101 via the communication module 490, the first server 1120 may transmit the healthcare program to the electronic device 1101 through the communication module 490.

Meanwhile, according to an embodiment of the disclosure, the electronic device 1101 may request the healthcare program from the first server 1120 using the communication module 490, and the first server 1120 may determine whether the healthcare program requested by the electronic device 1101 exists. When the requested healthcare program exists, the first server 1120 may transmit the requested healthcare program to the electronic device 1101 through the communication module 490. When the requested healthcare program does not exist, the first server 1120 may request the corresponding healthcare program from the second server 470 (shown in FIG. 4), and may receive the healthcare program from the second server 470 and may transmit the healthcare program to the electronic device 1101 through the communication module 490. However, the disclosure is not limited thereto.

In operation 1103, in an embodiment, the processor 430 (shown in FIG. 4) may generate a schedule based on the healthcare program received via the communication module 490. For example, the processor 430 may analyze the received healthcare program to extract healthcare program information, session information, activity information, and the like, and may generate a schedule based on the extracted healthcare program information, session information, and activity information. The generated schedule may include at least one activity, a span during which to perform an activity, a frequency, the days during the span during which to perform the activity, repetition counts, and the like.

In operation 1104, in an embodiment, the processor 430 may provide a notification via the display 420, the audio module, or the motor based on the generated schedule. That is, the processor 430 may display the notification on the display 420 of the electronic device 410 and may provide the notification to the user using voice, vibration, or the like. In an embodiment, the user of the electronic device 1101 may receive the notification via the display 420 at the time at which the activity included in the healthcare program needs to be performed. Also, in an embodiment, if the healthcare program does not include a specific time at which the activity is to be performed, the processor 430 may provide the notification at the time at which the user is able to perform the activity based on context information. The context information may include calendar information, user schedule information, SNS information, SMS information, weather information, holiday information, and the like. In one embodiment, if the healthcare program includes a specific time at which the activity is to be performed, the processor 430 may provide a notification prior to the specific time in order to enable preparation for the activity. For example, if the specific time at which the activity is to be performed is 14:00, the processor 430 may provide a first alarm at 12:00 and a second alarm at 13:00. The duration and frequency of the alarm may be changed in various ways.

In operation 1105, in an embodiment, the processor 430 may detect the activity of the user using the sensor module 450 (shown in FIG. 4). If the processor 430 does not detect the activity of the user using the sensor module 450, the processor 430 may provide the notification again via the display 420 based on the schedule. However, the disclosure is not limited thereto.

In operation 1106, in an embodiment, the processor 430 may link the activity included in the schedule with the item of the exercise application when the activity of the user is detected using the sensor module 450. Also, in an embodiment, the processor 430 may detect activity information of the user using the audio module or the camera module. Unlike this, the processor 430 may receive the activity information of the user through the audio module or the camera module, and the received activity information may be reflected in the item of the exercise application. In addition, in an embodiment, the processor 430 may receive the activity information via an input device such as a touch panel, and may also receive the activity information through the user's touch input. However, the disclosure is not limited thereto.

In operation 1107, in an embodiment, the processor 430 may reflect the activity information of the user including the activity result of the user analyzed according to the detection of the activity of the user in the item of the exercise application. Therefore, the activity information of the user including the activity result of the user may be stored in a memory 4400 through the item of the exercise application without any additional input, and the user may confirm the activity result of the user by browsing the item of the exercise application. However, the disclosure is not limited thereto.

In operation 1108, in an embodiment, the processor 430 may perform control to store healthcare program progress information in the memory 440 (shown in FIG. 4). The healthcare program progress information may include user activity information and may include a healthcare program progress status of the user performed according to the schedule. However, the disclosure is not limited thereto.

In operation 1109, in an embodiment, the processor 430 may transmit the healthcare program progress information to the first server 1120 using the communication module 490. However, the disclosure is not limited thereto, and the healthcare program progress information may be transmitted through the communication module 490 before completion of the healthcare program or upon completion of the healthcare program.

In operation 1110, in an embodiment, the processor 430 may detect user feedback input according to the progress of the healthcare program of the user. The user may input feedback for the healthcare program while the healthcare program is in progress or after completion of the healthcare program. On the other hand, if there is no user feedback, the processor 430 may not perform operation 1110.

Meanwhile, according to an embodiment of the disclosure, the processor 430 may provide a notification requesting feedback through the display 420 when there is no feedback input while the healthcare program is in progress according to the schedule. For example, the processor 430 may display a feedback request notification on the display 420, may request feedback by voice through the audio module, and may request feedback by vibration through the motor. However, the disclosure is not limited thereto.

In operation 1111, in an embodiment, when the feedback input of the user is detected, the processor 430 may store the user feedback related to the healthcare program in the memory 440, and may transmit the feedback to the first server 1120 using the communication module 490. The user feedback may include an assessment of the activity included in the healthcare program, a request for advice on activities, and so on. The processor 430 may transmit the feedback information to the first server 1120 in real time while the healthcare program is in progress, using the communication module 490 or may transmit all of the feedback information together after completion of the healthcare program. However, the disclosure is not limited thereto.

In operation 1112, in an embodiment, the first server 1120 may generate a service based on at least one of the healthcare program progress information and the feedback received from the electronic device 1101 through the communication module 490. For example, the first server 1120 may provide a service, such as a response to a question, through the communication module 490 in response to a request in the form of a user question or the like. The response to the user question may be generated by the first server 1120 and may be provided through the communication module 490, the user question may be transmitted to an external electronic device of a provider, and a service, such as the response received from the provider, may be provided to the electronic device 1101 through the communication module 490. However, the disclosure is not limited thereto.

Meanwhile, according to an embodiment of the disclosure, the first server 1120 may update the healthcare program based on at least one of the healthcare program progress information and the feedback received from the electronic device 1101 through the communication module 490. The first server 1120 may receive the healthcare program progress information through the communication module 490 to compare a target value and an accomplishment value of the user, and may transmit the healthcare program, the target value of which is modified, to the electronic device 1101 through the communication module 490. That is, if the user has already achieved a weight reduction of 5 kg which is the target value suggested by a weight management healthcare program in the middle of the healthcare program schedule, a healthcare program including more intense exercise or the like may be provided to the electronic device 1101 by modifying the target value to 8 kg.

Meanwhile, according to an embodiment of the disclosure, the first server 1120 may generate a message for healthcare program modification based on the at least one of the healthcare program progress information and the user feedback. The first server 1120 may transmit the message for the healthcare program modification to the electronic device 1101 via the communication module 490. The electronic device 1101 may receive the message for healthcare program modification from the first server 1120 via the communication module 490, and may correct at least a portion of the healthcare program based on the message for healthcare program modification. However, the disclosure is not limited thereto.

Meanwhile, according to an embodiment of the disclosure, the electronic device 1101 may modify the healthcare program by additionally reflecting user's profile information as well as the healthcare program progress information or the feedback of the user. The user's profile information may include body weight, body fat, height, age, and the like. However, the disclosure is not limited thereto.

In operation 1113, in an embodiment, the processor 430 may receive the updated healthcare program and service from the first server 1120 using the communication module 490. Accordingly, the processor 430 may update the content of the healthcare program, may provide a notification for the updated healthcare program, and may display the notification via the display 420 so that the user can view the service. However, the disclosure is not limited thereto.

In operation 1114, in an embodiment, the processor 430 may detect completion of the healthcare program. The completion of the healthcare program may be determined by the processor 430 when the end date of the generated schedule is detected, and may be determined by a user's input. However, the disclosure is not limited thereto.

In operation 1115, in an embodiment, the processor 430 may transmit healthcare program completion information via the communication module 490. The healthcare program completion information may include information about completion of the healthcare program, the degree of achievement of the healthcare program, and a healthcare program progress result of the user. However, the disclosure is not limited thereto.

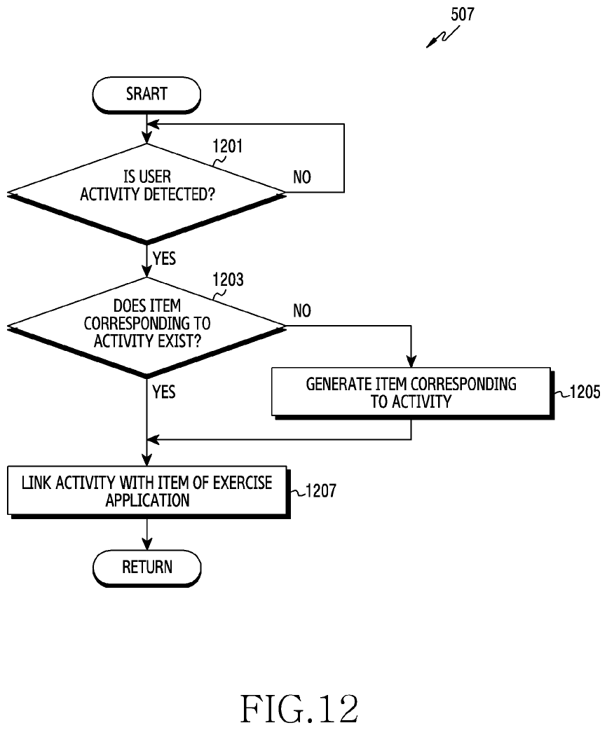

FIG. 12 is a flowchart illustrating an operation method of an electronic device according to an embodiment of the disclosure.

Operation 507 of FIG. 5 will be described in detail with reference to FIG. 12. Referring to FIG. 12, in operation 1201, in an embodiment, the processor 430 may use the sensor module 450 to detect user activity. When no user activity is detected, the processor 430 may confirm in real time whether the user activity is detected. However, the disclosure is not limited thereto.

In operation 1203, in an embodiment, when the user activity is detected using the sensor module 450, whether an item corresponding to the activity included in a schedule exists in an exercise application may be confirmed. For example, as shown in FIG. 12, the processor 430 may confirm whether the corresponding activity exists in the item of the exercise application. The item of the exercise application may be software that includes detailed conditions and the like so that each specific activity of the user can be sensed and detected. However, the disclosure is not limited thereto.

In operation 1205, in an embodiment, when there is no item corresponding to the activity included in the schedule in the exercise application, the processor 430 may generate the item corresponding to the activity. The generated item may include a value that is sensed while the user activity is detected, and may track performance of the activity of the generated item by the user when the same sensing value is detected in the future. However, the disclosure is not limited thereto.

In operation 1207, in an embodiment, when the item corresponding to the activity included in the schedule exists in the exercise application or when the item corresponding to the activity is generated, the processor 430 may link the activity with the item of the exercise application. Therefore, when the user performs the same activity thereafter, the linked item may sense and track the activity. However, the disclosure is not limited thereto.

In an embodiment, operation 509 of FIG. 5 may be performed (return) after the completion of operation 1207. However, the disclosure is not limited thereto.

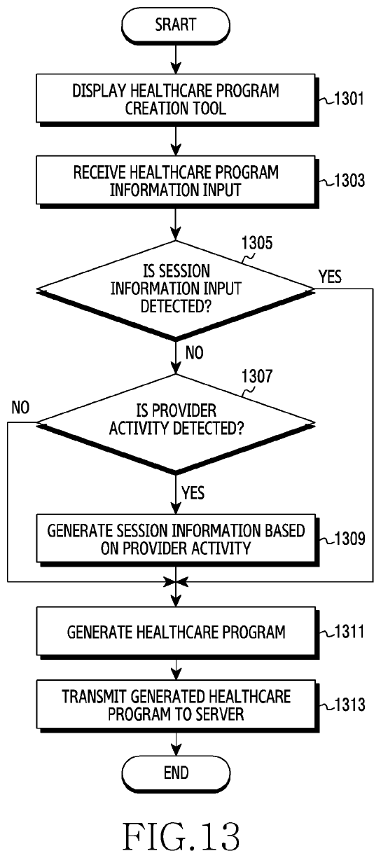

FIG. 13 is a flowchart illustrating a method of generating a healthcare program according to an embodiment of the disclosure.

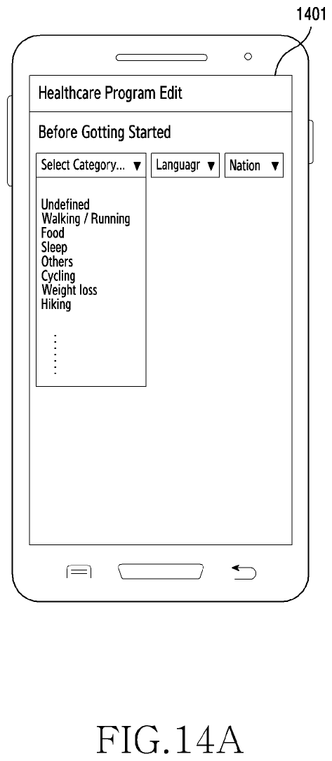

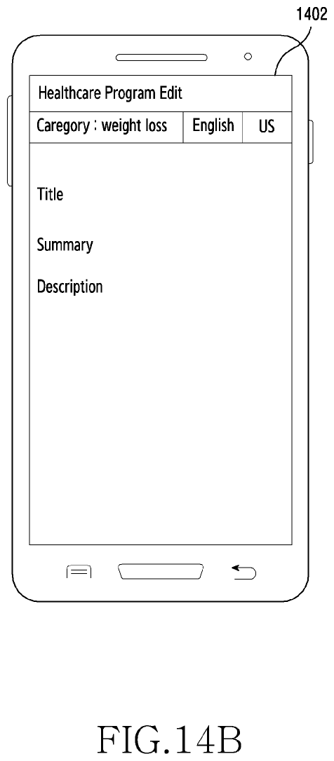

FIGS. 14A and 14B are exemplary diagrams illustrating a method of generating a healthcare program according to an embodiment of the disclosure.

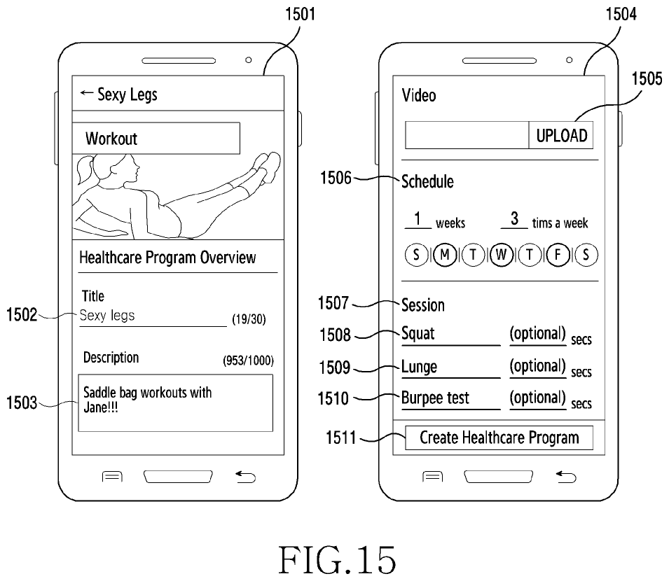

FIG. 15 is an exemplary diagram illustrating a method of generating a healthcare program according to an embodiment of the disclosure.

Referring to FIG. 13, the electronic device 410 (shown in FIG. 4) may be a user's electronic device, and may be an electronic device of a provider. It is assumed in FIG. 13 that the electronic device 410 is the electronic device of a provider. In operation 1301, in an embodiment, the processor 430 (shown in FIG. 4) may display a healthcare program creation tool via the display 420. The healthcare program creation tool may provide a user interface for inputting healthcare program information, session information, activity information, and the like of the healthcare program to be generated. However, the disclosure is not limited thereto.

Meanwhile, according to an embodiment of the disclosure, the provider of the electronic device may generate the healthcare program in a separate user interface, such as a web page provided by the first server 460 (shown in FIG. 4). For example, referring to FIGS. 14A and 14B, the first server 460 may provide the healthcare program creation tool including a user interface that can be used by a healthcare program provider through the communication module 490. The healthcare program provider may easily generate, distribute, and manage the healthcare program using the healthcare program creation tool provided by the first server 460 through the communication module 490. The healthcare program generated by the healthcare program provider may be stored in the first server 460 via the communication module 490 and may be provided to the user of the electronic device 410 through the communication module 490. For example, in the web page 1401 provided by the first server 460, the healthcare program provider may select the category, language, and country of the healthcare program to be registered in a program edit menu. The healthcare program provider may input metadata, session information, activity information, and the like of the healthcare program into the title, summary, and description in the healthcare program edit page 1402. The metadata of the healthcare program may include a healthcare program title, description, duration, effect, intensity, target user, and the like. For example, the title of the healthcare program is “making beautiful legs”, and the description is “challenge a celebrity's legs with two weeks of exercise”, the duration is “two weeks”, the effect is “strengthening lower-body muscle and tone up”, the intensity is “medium”, and the target user is “beginner”. The session information may include information on when to perform the session together with the title and the description, and may include the activity information included in the session. For example, the title in the session information is “strengthening the lower-body muscle”, the description is “strengthen the lower-body through squat and lunge exercises”, the period is “every Monday, Wednesday, and Friday”, and the related activity is “activities 1 to 4”. The activity information may include the details of each activity, goal information, items of associated exercise applications, and the like. For example, activity 1 may be “Squat/item: squat/goal: time (60 seconds) or number (20 counts)”, activity 2 may be “Lunge/item: lunge/goal: time (60 seconds) or number (20 counts)”, activity 3 may be “weight training/item: weight/goal: 0.5 kg increase in amount of muscle mass”, and activity 4 may be “food intake/item: food/target: calories (1500)”. However, the disclosure is not limited thereto.

In operation 1303, in an embodiment, the processor 430 may receive an input of the healthcare program information via the input device. The healthcare program information may include the title, description, duration, organization, expert information, etc., of the healthcare program. For example, referring to FIG. 15, the electronic device 410 may display the healthcare program creation tool 1501 provided by the first server 460 via the communication module 490, through the display 420, and may detect an input of the provider in a title 1502 and a description 1503 of the healthcare program creation tool 1501 using the input device of the provider or the sensor module 450. For example, the provider may input “sexy legs” in the title, and “saddle bag workouts with Miley Cyrus” in the description. However, the disclosure is not limited thereto.

In operation 1305, in an embodiment, the processor 430 may confirm whether the input of the session information is detected through the input device or the sensor module 450. For example, referring to FIG. 15, in order to generate a schedule in the healthcare program, the healthcare program provider may write the activity frequency in a schedule item 1506 of a healthcare program detail page 1504, and may additionally input the title, the number of activities 1508, 1509, and 1510, and the like in a session 1507. In addition, the user may upload a video captured by himself or herself using an upload menu 1505. When the input of the session information is detected, the processor 430 may receive an input of a healthcare program generation button 1511 of the user and may generate the healthcare program in operation 1311. However, the disclosure is not limited thereto.

In operation 1307, in an embodiment, the processor 430 may detect the activity of the provider in the case in which an input of the session information is not detected at the input device or the sensor module 450. In the case in which the input of the session information and the activity of the provider are not detected, the processor 430 may generate a healthcare program that does not include the session information, but may continue to display the healthcare program creation tool to detect the input of the session information. However, the disclosure is not limited thereto.

In operation 1309, in an embodiment, the processor 430 may generate the session information based on the detected provider activity. For example, referring to FIG. 15, in order to generate the schedule in the healthcare program, the provider may additionally input a title, a frequency, and the number of each activity. However, unlike this, if the provider actually performs the activity to be performed while holding the electronic device 410 with the body, the processor of the electronic device 410 may detect the activity of the provider to determine the title, frequency, and the number of the activities. That is, the healthcare program provider may include the title, frequency, and the number of activities performed by the provider in the healthcare program without having to input the title, frequency, or number of the activities. In addition, the title, frequency, and the number of activities of the healthcare program provider may be detected through a separate wearable device. However, the disclosure is not limited thereto.

In operation 1311, in an embodiment, the processor 430 may generate the healthcare program based on the input healthcare program information and the generated session information. In operation 1313, in an embodiment, the processor 430 may transmit the healthcare program generated via the communication module 490 to the server. Unlike this, when the healthcare program is generated according to the user interface provided by the first server 460, operation 1313 may be omitted. However, the disclosure is not limited thereto.

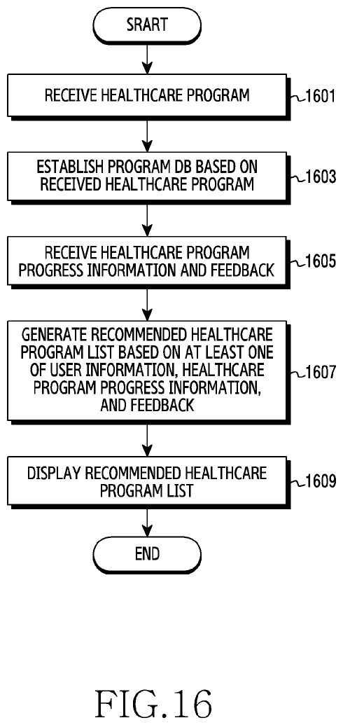

FIG. 16 is a flowchart illustrating an operation method of a server according to an embodiment of the disclosure.

Referring to FIG. 16, in operation 1601, in an embodiment, the first server 460 (shown in FIG. 4) may receive at least one healthcare program from the electronic device 410 (shown in FIG. 4) via the communication module 490. The first server 460 may receive user information or provider information from the electronic device 410 through the communication module 490 or may provide a user interface such as a separate web page to receive the user information or the provider information. The first server 460 may store the healthcare program, the user information, the provider information, and the like. The user information may include user behavior information such as a user profile, a healthcare program search, and the like described above. However, the disclosure is not limited thereto, and the first server 460 may store a variety of information such as a user's healthcare program preference, health status, and the like.

In operation 1603, in an embodiment, the first server 460 may establish a healthcare program database based on the received healthcare program, user information, and provider information. The first server 460 may classify the healthcare program according to criteria such as age, gender, and area based on accumulated healthcare program data and the like. However, a variety of data can be utilized without being limited thereto.

In operation 1605, in an embodiment, the first server 460 may receive at least one of the healthcare program progress information and the feedback information from the electronic device 410 through the communication module 490. The first server 460 may record the at least one of the healthcare program progress information and the feedback in the healthcare program database. The healthcare program progress information may include the result of each activity included in the healthcare program, whether the entire schedule is completed, and the like. The feedback of the user may include comments on each activity, an advice request, a healthcare program improvement plan, and the like. However, the disclosure is not limited thereto.

In operation 1607, in an embodiment, the first server 460 may generate a recommended healthcare program list specified for each user based on the accumulated healthcare program data, the context information (e.g., seasons), the user information, the healthcare program progress information, and the feedback. For example, the first server 460 may recommend a healthcare program that many women in their 30s have subscribed to, in consideration of age, gender, and the number of healthcare program subscriptions, may recommend a healthcare program having a high grade for men in their 20s in consideration of age, gender, and grade. Also, the first server 460 may recommend a healthcare program such as “weight reduction of 5 kg within fifteen days” to women in their 40s having a weight of 60 to 70 kg, in consideration of age, weight and gender, may recommend a healthcare program such as “making a bikini body” to women in their 20s or 30s in the summer in consideration of season, age, and gender, and may recommend healthcare such as “making moist skin” in the winter in consideration of seasons. Also, the first server 460 may recommend a healthcare program in a category with many clicks in consideration of the user's clicks and search information, and may recommend a low intensity or high intensity healthcare program based on the at least one of the healthcare program progress information and the feedback information of the user or recommend a one-to-one customized healthcare program or a healthcare program that can be performed independently. Also, the first server 460 may recommend a health care program that is most frequently clicked today or a healthcare program that is most frequently subscribed today based on real time information. However, the disclosure is not limited thereto.

In operation 1609, in an embodiment, the first server 460 may display, on the display 420, the recommended healthcare program list via a web page or a separate application. For example, the first server 460 may display, on the display 420, the recommended healthcare program list in the separate web page or the like, and may transmit a notification message indicating that the recommended healthcare program list has been updated or a URI to the electronic device 410 via the communication module 490. In addition, unlike this, the first server 460 may transmit the recommended healthcare program list in the form of a text message or a notification message through the communication module 490, and may transmit the recommended healthcare program list so that the recommended healthcare program list can be displayed in the exercise application of the electronic device 410 through the communication module 490. However, the disclosure is not limited thereto.

Meanwhile, the first server 460 may continuously update the recommended healthcare program list through the communication module 490 even after the completion of operation 1609. However, the disclosure is not limited thereto.

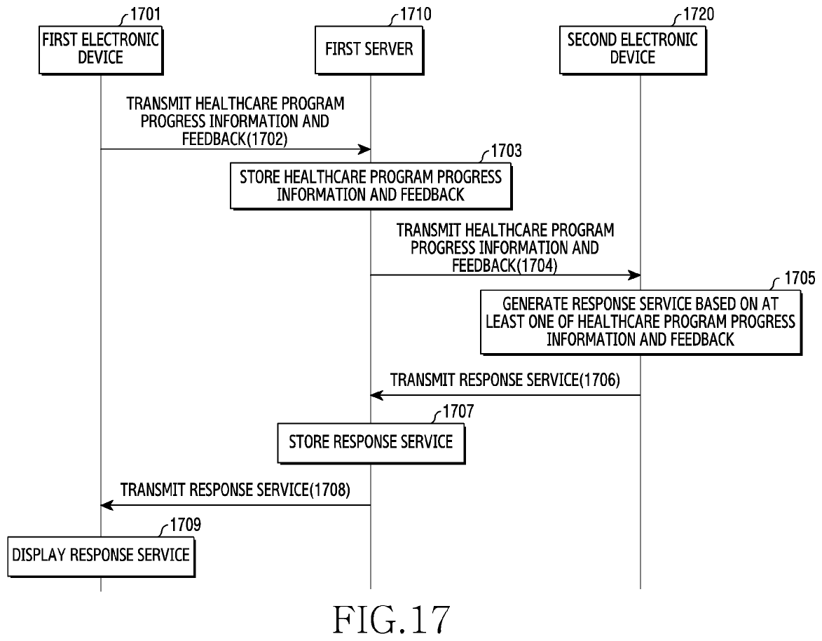

FIG. 17 is a flowchart illustrating a system among a first electronic device, a server, and a second electronic device according to an embodiment of the disclosure.

Referring to FIG. 17, in operation 1702, in an embodiment, a first electronic device 1701 may transmit at least one of healthcare program progress information and feedback to a first server 1710 through the communication module 490. Here, the first electronic device 1701 may refer to an electronic device of a user. However, the disclosure is not limited thereto.

In operation 1703, in an embodiment, the first server 1710 may store the at least one of the healthcare program progress information and the feedback which are received from the first electronic device 1701. The first server 1710 may record the at least one of the received healthcare program progress information and feedback in a healthcare program database. However, the disclosure is not limited thereto.

In operation 1704, in an embodiment, the first server 1710 may transmit the at least one of the healthcare program progress information and the feedback to a second electronic device 1702. Here, the second electronic device 1720 may be an electronic device of a provider of the healthcare program associated with the healthcare program progress information and the feedback. However, the second electronic device 1720 may be an electronic device of an expert of the healthcare program associated with the healthcare program progress information and the feedback, without being limited thereto.

In operation 1705, in an embodiment, the second electronic device 1720 may generate a response service based on the at least one of the healthcare program progress information and the feedback. For example, the second electronic device 1720 may receive an input of a response message from the provider or the expert, and may generate a response service including the received response message. The type of the response service may be configured to be displayable in the first electronic device 1701. However, the disclosure is not limited thereto.

In operation 1706, in an embodiment, the second electronic device 1720 may transmit the response service to the first server 1710 through the communication module 490. Unlike this, the second electronic device 1720 may directly transmit the response service to the first electronic device 1701 through the communication module 490. However, the disclosure is not limited thereto.

In operation 1707, in an embodiment, the first server 1710 may store the response service received at the second electronic device 1720 through the communication module 490, may record the same in the healthcare program database, and may utilize the same as reference data. However, the disclosure is not limited thereto.

In operation 1708, in an embodiment, the first server 1710 may transmit the response service to the first electronic device through the communication module 490. In operation 1709, in an embodiment, the first electronic device 1701 may display the received response service through the display 420. For example, the first electronic device 1701 may display the response service in the form of a notification in a separate user interface through the display 420. Alternatively, the first electronic device 1701 may display the response service in the exercise application. However, the disclosure is not limited thereto.

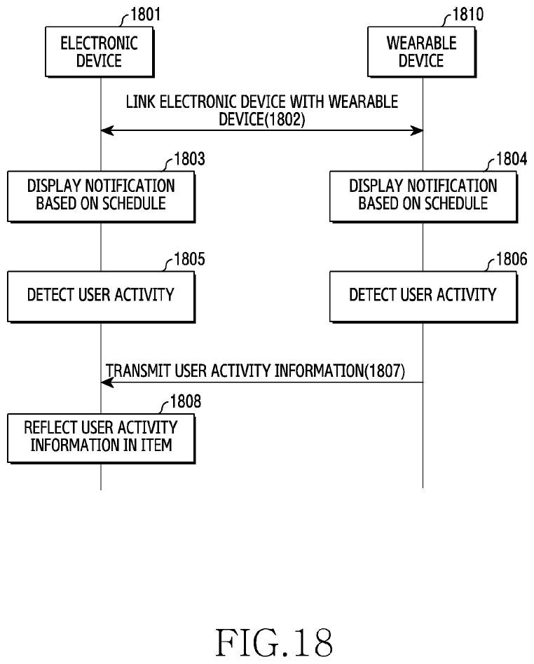

FIG. 18 is a flowchart illustrating a system between an electronic device according to an embodiment of the disclosure and a wearable device.

Referring to FIG. 18, in operation 1802, in an embodiment, an electronic device 1801 and a wearable device 1810 may be linked with each other. For example, the electronic device 1801 may be connected to the wearable device 1810 via a local area communication method such as Wi-Fi, Bluetooth, Bluetooth low energy (BLE), near-field communication (NFC), and the like. However, the disclosure is not limited thereto.

In operation 1803, in an embodiment, the electronic device 1801 may display a notification based on a schedule generated through the display 420. At the same time or before or after this, in operation 1804, the wearable device 1810 may display a notification based on the schedule generated in the electronic device 1801. A user may confirm a time to perform a corresponding activity through the electronic device 1801 or the wearable device 1810. However, the disclosure is not limited thereto.

In operations 1805 and 1806, the electronic device 1801 and the wearable device 1810 may detect a user activity using the sensor module 450. In operation 1807, in an embodiment, the wearable device 1810 may transmit user activity information through the communication module 490. An operation in which the wearable device 1810 detects the user activity may be an operation to assist the electronic device 1801. For example, the electronic device 1801 may detect the user activity through the wearable device 1810 while detecting the user activity through the sensor module 450. That is, the wearable device 930 may be an auxiliary means for accurately detecting the user activity. In operation 1808, in an embodiment, the electronic device 1801 may reflect the detected user activity information in the item of the exercise application. However, the disclosure is not limited thereto.

A method according to various embodiments of the disclosure may include: receiving a first healthcare program from another electronic device; generating a first schedule including at least one activity based on the received first healthcare program; providing a notification to a user based on the first schedule; sensing an activity of the user related to a first activity during the at least one activity; linking, with the first activity, a first item included in an exercise application associated with the first activity; and reflecting, in the first item, activity information of the user related to the first activity.

According to an embodiment, the method may further include detecting first healthcare program information, session information, and activity information based on the received first healthcare program.

According to an embodiment, the generating the first schedule may include generating the first schedule based on the session information and the activity information.

According to an embodiment, the method may further include: receiving a second healthcare program from the other electronic device; generating a second schedule including at least one activity based on the received second healthcare program; providing a notification to the user based on the second schedule; sensing an activity of the user related to a second activity during the at least one activity; and linking the second activity with the first item when the second activity overlaps the first activity.

According to an embodiment, the method may further include: storing first healthcare program progress information including a first healthcare program progress status of the user according to the user activity information and the first schedule.

According to an embodiment, the method may further include: detecting feedback of the user associated with the first healthcare program; and transmitting at least one of the first healthcare program progress information and the feedback of the user to the other electronic device.

According to an embodiment, the method may further include: receiving a service generated based on the at least one of the first healthcare program progress information and the feedback of the user from the other electronic device.

According to an embodiment, the linking the first activity with the item of the exercise application may further include: determining whether an item corresponding to the first activity exists in the exercise application; generating an item corresponding to the first activity when the item corresponding to the first activity does not exist in the exercise application; and linking the generated item with the first activity.

According to an embodiment, the method may further include: determining whether the first healthcare program is completed based on the activity information of the user reflected in the first item of the exercise application.

A method according to various embodiments of the disclosure may include: receiving an input of healthcare program information; determining whether an input of session information is detected; determining whether an activity of a user is detected when the input of the session information is not detected; generating the session information based on the activity of the user when an activity of a provider is detected; and generating a healthcare program based on the healthcare program information and the session information.

The disclosure has been discussed above in connection with the exemplary embodiments thereof. It will be understood by those skilled in the art to which the disclosure belongs that the disclosure may be implemented in modified forms without departing from the essential characteristics of the disclosure. Therefore, the embodiments disclosed herein should be considered from an illustrative point of view, rather than a limitative point of view. The scope of the disclosure is found not in the above description but in the accompanying claims, and all differences falling within the scope equivalent to the claims should be construed as being included in the disclosure.

1. An operation method of an electronic device, the operation method comprising:
receiving a first healthcare program from another electronic device;
generating a first schedule including at least one activity based on the received first healthcare program;
providing a notification to a user based on the first schedule;
sensing an activity of the user related to a first activity during the at least one activity;
linking, with the first activity, a first item included in an exercise application associated with the first activity; and
reflecting, in the first item, activity information of the user related to the first activity.

2. The operation method of claim 1, further comprising:
detecting first healthcare program information, session information, and activity information based on the received first healthcare program.

3. The operation method of claim 2, wherein the generating the first schedule comprises generating the first schedule based on the session information and the activity information.

4. The operation method of claim 1, further comprising:
receiving a second healthcare program from the other electronic device;
generating a second schedule including at least one activity based on the received second healthcare program;
providing a notification to the user based on the second schedule;
sensing an activity of the user related to a second activity during the at least one activity; and
linking the second activity with the first item when the second activity overlaps the first activity.

5. The operation method of claim 1, further comprising:
storing first healthcare program progress information including a first healthcare program progress status of the user according to the user activity information and the first schedule.

6. An electronic device comprising:
a communication module;
a sensor module;
a processor configured to be electrically connected to the communication module and the sensor module; and
a memory configured to be electrically connected to the processor,
wherein the memory is controlled to store, when executed, instructions that cause the processor to:
receive a first healthcare program from at least one another electronic device using the communication module,
generate a first schedule including at least one activity based on the received first healthcare program,
provide a notification to a user based on the first schedule,
sense an activity of the user related to a first activity during the at least one activity, using the sensor module,
link, with the first activity, a first item included in an exercise application associated with the first activity, and
reflect, in the first item, activity information of the user related to the first activity acquired using the sensor module.

7. The electronic device of claim 6, wherein the instructions cause the processor to detect at least one of first healthcare program information, session information, and activity information based on the received first healthcare program.

8. The electronic device of claim 7, wherein the instructions cause the processor to generate the first schedule based on the session information and the activity information.

9. The electronic device of claim 6, wherein the instructions cause the processor to
receive a second healthcare program from at least one server using the communication module,
generate a second schedule including at least one activity based on the received second healthcare program,
provide a notification to the user based on the second schedule,
sense an activity of the user related to a second activity during the at least one activity, using the sensor module, and
link the second activity with the first item when the second activity overlaps the first activity.

10. The electronic device of claim 6, wherein the instructions cause the memory to store first healthcare program progress information including a first healthcare program progress status according to the user activity information and the first schedule.

11. The electronic device of claim 10, wherein the instructions cause the processor to
detect feedback of the user associated with the first healthcare program, and
transmit at least one of the first healthcare program progress information and the feedback of the user to the other electronic device.

12. The electronic device of claim 11, wherein the instructions cause the processor to receive a service generated based on the at least one of the first healthcare program progress information and the feedback of the user from the other electronic device.

13. The electronic device of claim 6, wherein the instructions cause the processor to
determine whether an item corresponding to the first activity exists in the exercise application,
generate an item corresponding to the activity when the item corresponding to the first activity does not exist in the exercise application, and
link the generated item with the first activity.

14. The electronic device of claim 6, wherein the instructions cause the processor to determine whether the first healthcare program is completed based on first item of the exercise application.

15. An electronic device comprising:
a sensor configured to be functionally connected to the electronic device; and
a processor,
wherein the processor is configured to:
receive an input of healthcare program information,
determine whether an input of session information is detected,
determine whether an activity of a user is detected when the input of the session information is not detected,
generate the session information based on the activity of the user when the activity of the user is detected, and
generate a healthcare program based on the healthcare program information and the session information.

16. The operation method of claim 1, further comprising:
detecting feedback of the user associated with the first healthcare program, and
transmitting at least one of the first healthcare program progress information and the feedback of the user to the other electronic device.

17. The operation method of claim 16, further comprising:
receiving a service generated based on the at least one of the first healthcare program progress information and the feedback of the user from the other electronic device.

18. The operation method of claim 1, wherein the linking of the first item comprising:
determining whether the first item corresponding to the first activity exists in the exercise application;
generating the first item corresponding to the activity when the first item corresponding to the first activity does not exist in the exercise application, and
linking the generated first item with the first activity.

19. The operation method of claim 1, further comprising:
determining whether the first healthcare program is completed based on first item of the exercise application.
